(19) United States
(12) Patent Application Publication
(10) Pub. No.: US 2026/0226473 A1
(43) Pub. Date: Aug. 6, 2026

(54) METHODS FOR REDUCING THE RISK OF MAJOR ADVERSE CARDIOVASCULAR EVENTS IN PRIMARY PREVENTION SUBJECTS

(71) Applicant: NOVARTIS AG, Basel (CH)

(72) Inventors: Anastasia LESOGOR, Wenslingen (CH); Pierre MAHEUX, Québec (CA); Qing SHAO, Parsippany, NJ (US)

(21) Appl. No.: 19/151,559

(22) PCT Filed: Feb. 2, 2024
(86) PCT No.: PCT/IB2024/050978

§ 371 Date: Jul. 28, 2025

Related U.S. Application Data
(60) Provisional application No. 63/483,116, filed on Feb. 3, 2023.

(51) Int. Cl. C12N 15/113; A61J 1/05; A61K 9/00; A61K 9/08; A61P 3/06

(52) U.S. Cl. CPC C12N 15/1137; A61K 9/0019; A61K 9/08; A61P 3/06; A61J 1/05; C12N 2310/14; C12N 2310/315; C12N 2310/321; C12N 2310/322; C12N 2310/351

(57) ABSTRACT

The present invention relates to methods for reducing the risk of major adverse cardiovascular events (MACE) in subjects comprising administration of RNAi agents, RNAi agents and pharmaceutical compositions for use in such methods, and uses of an RNAi agent for the manufacture of a medicament for such methods. Provided herein are methods that are particularly effective in prevention of cardiovascular death, non-fatal myocardial infarction (MI), non-fatal ischemic stroke, or urgent coronary revascularization, in subjects that have not experienced a major atherosclerotic cardiovascular disease (ASCVD) event, especially where the subject is at increased risk for a first MACE.

### CROSS-REFERENCE TO RELATED APPLICATIONS

This application claims priority to, and the benefit of, U.S. Application No. 63/483,116, filed on Feb. 3, 2023, the content of which is incorporated herein by reference in its entirety.

### SEQUENCE LISTING

The instant application contains a Sequence Listing which has been submitted electronically in .XML format and is hereby incorporated by reference in its entirety. Said .XML copy, created on Feb. 1, 2024, is named PAT059477-WO-PCT_SL.xml and is 25 KB in size.

### FIELD OF THE INVENTION

The present invention relates to methods for reducing the risk of major adverse cardiovascular events (MACE) in subjects comprising administration of RNAi agents, and RNAi agents and pharmaceutical compositions for use in such methods. Provided herein are methods that are particularly effective in prevention of cardiovascular death, non-fatal myocardial infarction (MI), non-fatal ischemic stroke, or urgent coronary revascularization (4P-MACE), in subjects that have not experienced a major atherosclerotic cardiovascular disease (ASCVD) event, especially where the subject is at increased risk for a first MACE.

### BACKGROUND

Although there has been substantial improvement in recent decades, cardiovascular disease (CVD) remains the leading cause of death worldwide, resulting in over 17 million deaths annually (WHO 2019). In 2019, coronary events were expected to occur in 1,055,000 individuals in the United States (US), including 720,000 new coronary events and 335,000 recurrent coronary events (Benjamin et al 2019). These facts highlight the importance of preventing a first major ASCVD event in patients at high CV risk.

Long-term exposure to elevated low-density lipoprotein cholesterol (LDL-C) increases cumulative plaque burden and the risk of ASCVD, and higher lifelong exposure to LDL-C, especially long-term exposure to persistently elevated LDL-C, leads to acute coronary syndromes earlier in life and is a major risk factor for the development of ASCVD. There is therefore an unmet need to reduce the risk of major adverse cardiovascular events (MACE) in subjects, particularly where subjects have not experienced a major ASCVD event (also known as primary prevention), for example where subjects have not experienced a major ASCVD event within about 12 months prior to administration of the RNAi agent as described herein.

### SUMMARY OF THE INVENTION

Accordingly, the present invention addresses this need by providing a method of reducing the risk of a major adverse cardiovascular event (MACE) in a subject that has not experienced a major atherosclerotic cardiovascular disease (ASCVD) event,
wherein the MACE is cardiovascular death, non-fatal myocardial infarction (MI), non-fatal ischemic stroke, or urgent coronary revascularization,
wherein the method comprises administering an interfering ribonucleic acid (RNAi) agent to the subject, wherein the RNAi agent comprises:
an antisense strand comprising the nucleotide sequence 5′-asCfsaAfAfAfgCfaAfaAfcAfgGfuCfuagsasa-3′ (SEQ ID NO: 3), and
a sense strand comprising the nucleotide sequence 5′-csusagacCfuGfudTuugcuuuugu-3′ (SEQ ID NO: 4),
wherein a, g, c, and u are 2′-O-methyl (2′-OMe) modified A, G, C, and U nucleotides, respectively; Af, Gf, Cf, and Uf are 2′-fluoro A, G, C, and U modified nucleotides, respectively; dT is a deoxy-thymine nucleotide and s is a phosphorothioate linkage; and
wherein the sense strand is conjugated to at least one ligand.

The invention also provides an RNAi agent for use in such methods, and a pharmaceutical composition comprising an RNAi agent for use in such methods.

The invention also provides the use of an RNAi agent for the manufacture of a medicament for the methods of the invention.

The inventors have found that by administering an RNAi agent to a subject as defined in the claims (e.g. inclisiran), the risk of 4P-MACE (the first occurrence of CV death, non-fatal MI, non-fatal ischemic stroke, or urgent coronary revascularization) is reduced in subjects at high CV risk without a prior major ASCVD event and with LDL-C ≥70 mg/dL (1.81 mmol/L) but <190 mg/dL (4.91 mmol/L), which receive treatment with the RNAi agent. These methods are particularly useful in reducing the risk of 4P-MACE in subjects that are at an increased risk for a first MACE. The inventors have also found that by administering an RNAi agent to a subject as defined herein (e.g. inclisiran), the risk of 3P-MACE (CV death, non-fatal MI, or non-fatal ischemic stroke) or CV death is reduced in the same subjects.

### BRIEF DESCRIPTION OF THE DRAWINGS

Embodiments of the invention will be described, by way of example, with reference to the following drawings, in which:

FIG. 1 illustrates a summary of the screening algorithm for the screening strategy, as used in Example 1.

FIG. 2 is a diagram of the study design, as used in Example 1.

FIG. 3 illustrates the testing procedure for analysis of primary and secondary endpoints in the trial according to Example 1.

### DETAILED DESCRIPTION OF THE INVENTION

### Methods of Treatment and Prophylaxis

Cardiovascular disease (including ASCVD) or death are attributed to potentially modifiable risk factors. Elevated LDL-C is a major risk factor for the development of ASCVD in a subject and leads to the development of ASCVD. Atherosclerosis is a chronic, progressive disease. The accumulation of fatty and fibrous material in the innermost layer of arteries results in the formation of atherosclerotic plaques. ASCVD includes (1) coronary artery disease (ischemic heart disease); acute coronary syndromes (MI, unstable angina), and stable angina; (2) carotid artery and cerebral artery disease: ischemic stroke, transient ischaemic attack (TIA); and (3) peripheral artery disease.

Long-term exposure to elevated LDL-C increases cumulative plaque burden and the risk of ASCVD, and higher lifelong exposure to LDL-C, especially long-term exposure to persistently elevated LDL-C, leads to acute coronary syndromes earlier in life and is a major risk factor for the development of ASCVD. There is therefore an unmet need to reduce the risk of MACE in subjects, particularly where subjects have not experienced a major ASCVD event or where subjects have not experienced a major ASCVD within about about 12 months prior to administration of the RNAi agent. The present invention addresses this need.

In some embodiments, the LDL-C level in the subject is lowered after administration of the RNAi agent. As explained above, LDL-C is one of the most readily modifiable risk factors for ASCVD; however, despite widespread statin use, four in five patients do not reach guideline-recommended LDL-C targets. The inhibition of PCSK9 offers an efficacious approach to lower LDL-C.

In one aspect, the present invention provides methods of reducing the risk of a major adverse cardiovascular event (MACE) in a subject wherein the subject has not experienced a major atherosclerotic cardiovascular disease (ASCVD) event, the method comprising administering an interfering ribonucleic acid (RNAi) agent to the subject. The present invention is based, at least in part, on the use of small interfering RNA (siRNA) molecules (which are a type of RNAi agent) as a means to reduce levels of PCSK9 protein, which in turn lowers LDL-C levels. Through RNA interference, the siRNA molecules bind intracellularly to the RNA-induced silencing complex (RISC), which enables it to cleave messenger RNA (mRNA) that encodes PCSK9. The cleaved mRNA is degraded and therefore unavailable for protein translation, resulting in decreased levels of the PSCK9 protein and, consequently, decreased levels of LDL-C. The inventors have found that the administration of an RNAi agent to lower LDL-C levels in a subject that has not experienced a major ASCVD event is effective in reducing the risk of 4P-MACE in the subject. 4P-MACE is defined as a composite of cardiovascular death, non-fatal myocardial infarction (MI), non-fatal ischemic stroke, or urgent coronary revascularization (i.e. the need for the subject to receive urgent coronary revascularization, for example in a subject with coronary artery disease (CAD)). As the skilled person will be aware, in the context of this invention, a ‘composite’ means that any one of the specified component events occurs.

Accordingly, the present invention provides a method of reducing the risk of a major adverse cardiovascular event (MACE) in a subject that has not experienced a ASCVD event, wherein the MACE is cardiovascular death, non-fatal myocardial infarction (MI), non-fatal ischemic stroke, or urgent coronary revascularization, wherein the method comprises administering an interfering ribonucleic acid (RNAi) agent as described herein to the subject.

### Major Adverse Cardiovascular Events (MACE)

As will be apparent to the person skilled in the art, reducing the risk of an event, for example a major adverse cardiovascular event (MACE), in a subject can also be described as:
(a) a method of preventing an event (e.g. a MACE) in a subject;
(b) a method of primary prevention for an event (e.g. a MACE) in a subject;
(c) a method of reducing the rate of occurrence of an event (e.g. a MACE) in a subject; or
(d) a method of delaying the occurrence of an event (e.g. a MACE) in a subject.

All references described herein to ‘reducing the risk’ of an event can also be described as ‘preventing’ such an event in all aspects of the invention, as is also apparent to the person skilled in the art.

As is also apparent to the skilled person, reducing the risk of an event in a subject is typically assessed relative to subjects that have not been administered the RNAi agent, e.g. subjects that are receiving placebo.

The methods of the invention, which comprise administering the RNAi agent to a subject, are effective in reducing the risk of a MACE in a subject that has not experienced a major atherosclerotic cardiovascular disease (ASCVD) event, compared to a subject that has not been administered said RNAi agent.

In some aspects, the risk of 4P-MACE is reduced. 4P-MACE is a composite of cardiovascular death, non-fatal myocardial infarction (MI), non-fatal ischemic stroke, or urgent coronary revascularization. Reducing the risk of 4P-MACE means reducing the risk of any one of these events in a subject.

The invention also provides methods of reducing the risk of cardiovascular death, non-fatal myocardial infarction (MI), non-fatal ischemic stroke, or urgent coronary revascularization in a subject.

The invention also provides methods of reducing the risk of other types of MACE in a subject that has not experienced a major atherosclerotic cardiovascular disease (ASCVD) event. For example, the inventors have found that by administering an RNAi agent to a subject as defined herein (e.g. inclisiran), the risk of 3P-MACE (cardiovascular death, non-fatal myocardial infarction, or non-fatal ischemic stroke) or the risk of cardiovascular death is reduced.

Reducing the risk of a MACE in a subject can be quantified using the hazard rates of a MACE (e.g. 4P-MACE or 3P-MACE). The hazard rate of a MACE is the probability that if the MACE has not already occurred, it will occur in the next time interval, divided by the length of that interval.

Reducing the risk of a MACE in a subject can also be quantified by determining the average time to the occurrence of a MACE (e.g. 4P-MACE or 3P-MACE) in subjects that have been administered the RNAi agent according to the methods of the invention, compared to the average time to the occurrence of a MACE in subjects that have not been administered the RNAi agent. For example, if the average time to the occurrence of a MACE in subjects that have been administered the RNAi agent according to the methods of the invention is greater than the average time to the occurrence of a MACE in subjects that have not been administered the RNAi agent, then the risk of a MACE is reduced.

In some embodiments, reducing the risk of a MACE in a subject that has not experienced a major atherosclerotic cardiovascular disease (ASCVD) event, wherein the MACE is cardiovascular death, non-fatal myocardial infarction (MI), non-fatal ischemic stroke, or urgent coronary revascularization, is defined by a reduction in the hazard rate in subjects that have been administered the RNAi agent, compared to subjects that have not been administered the RNAi agent. In some embodiments, the risk is defined by about a 20% reduction in the hazard rate in subjects that have been administered the RNAi agent, compared to subjects that have not been administered the RNAi agent.

### Reducing the Risk of Other Events

In further aspects, which can be combined with any of the other aspects described herein, the invention also provides methods of reducing the risk of an event, as further described below, in a subject that has not experienced a major atherosclerotic cardiovascular disease (ASCVD) event. These methods comprise administering the RNAi agent as described herein to a subject, and are effective in reducing the risk of these events in a subject that has not experienced a major atherosclerotic cardiovascular disease (ASCVD) event, compared to a subject that has not been administered said RNAi agent.

Exemplary events in accordance with these aspects of the invention are described below. The skilled person is able to determine whether the risk of the event is reduced, for example by determining the average time to the first occurrence of said event in subjects that have been administered the RNAi agent according to the invention, compared to the average time to the first occurrence of said event in comparable subjects that have not been administered the RNAi agent. These methods comprise administering the RNAi agent as described herein to a subject.

In some aspects, the invention provides methods of reducing the risk of CV events, for example first or recurrent non-fatal MI, non-fatal ischemic stroke, urgent revascularization, hospitalizations for unstable angina, heart failure hospitalization, or major adverse limb events (MALE).

In some aspects, the invention provides methods of reducing the risk of urgent coronary revascularization.

In some aspects, the invention provides methods of reducing the risk of all-cause mortality.

In some aspects, the invention provides methods of reducing the risk of CV death, non-fatal MI, non-fatal ischemic stroke, and urgent coronary revascularization (first or recurrent).

In some aspects, the invention provides methods of reducing the risk of CV death, non-fatal MI, and non-fatal ischemic stroke (first or recurrent).

In some aspects, the invention provides methods of reducing the risk of any coronary heart disease (CHD) event, for example wherein the CHD event is death from CHD, non-fatal MI, unstable angina requiring hospitalization, or urgent coronary revascularization.

In some aspects, the invention provides methods of reducing the risk of CV death.

In some aspects, the invention provides methods of reducing the risk of fatal or non-fatal MI.

In some aspects, the invention provides methods of reducing the risk of fatal or non-fatal ischemic stroke.

In some aspects, the invention provides methods of reducing the risk of all-cause mortality, non-fatal MI, or non-fatal ischemic stroke.

In some aspects, the invention provides methods of reducing the risk of all-cause mortality, non-fatal MI, non-fatal ischemic stroke, or urgent coronary revascularization.

In some aspects, the invention provides methods of reducing the risk of major adverse limb events (MALE), for example wherein the MALE is acute lower limb ischemia, lower limb amputation due to ischemia, or urgent lower limb revascularization for ischemia.

In some aspects, the invention provides methods of reducing the risk of first or recurrent all-cause revascularizations, for example wherein the revascularization is coronary, carotid, or peripheral.

In some aspects, the invention provides methods of reducing the risk of first or recurrent total coronary events, for example CHD death, MI, hospitalizations for unstable angina, and/or urgent/elective revascularizations.

In some aspects, the invention provides methods of reducing the risk of first or recurrent all-cause CV hospitalizations, for example hospitalizations for MI and hospitalizations for unstable angina.

In some aspects, the invention provides methods of reducing the risk of first or recurrent venous thromboembolisms resulting in hospitalization or death.

In a further aspect, the invention provides methods of reducing the level of LDL-C and other lipid parameters in a subject compared to baseline. For example, the lipid parameters may include one or more of: total cholesterol, high-density lipoprotein cholesterol (HDL-C), very low-density lipoprotein cholesterol (VLDL-C), non-high-density lipoprotein cholesterol (non-HDL-C), triglycerides and lipoprotein(a) (Lp(a)). The reduction in the level of LDL-C or other lipid parameters in a subject can be determined by comparing the percentage change or absolute change in the levels of LDL-C and other lipid parameters compared to baseline at various time points, for example the time points (e.g. V1, V2, V3, etc.) indicated in the Assessment Schedule (Schedule of Activities) in Example 1.

In exemplary embodiments of all aspects of the invention, the risk of adverse events (AEs) or severe adverse events (SAEs) in subjects that have been administered the RNAi agent is low. For example, the risk of AEs or SAEs in subjects that have been administered the RNAi agent is low if the rate or total occurrence of AEs or SAEs in subjects that have been administered the RNAi agent according to the invention is similar to or less than the rate or total occurrence of AEs or SAEs in subjects that have not been administered the RNAi agent, for example subjects that have been administered placebo.

### Reduction of LDL-C and PCSK9 Levels

In some embodiments of the invention, the administration of the RNAi agent reduces the LDL-C level in the subject. In exemplary embodiments, administration of the RNAi agent reduces the level of LDL-C in the subject, as compared to a baseline LDL-C level, by greater than about 20%, or by greater than about 25%, or by greater than about 30%, or by greater than about 35%, or by greater than about 40%, or by greater than about 45%, or by greater than about 50%, or by greater than about 55%, or by greater than about 60%.

In some embodiments, the reduction in the level of LDL-C after the RNAi agent is administered may be maintained for, at, or through about 30 days or more, 60 days or more, 90 days or more, 180 days or more, 1 year or more, 2 years or more, 3 years or more, 4 years or more, 5 years or more, or 6 years or more.

In an exemplary embodiment, these reductions in the level of LDL-C can be achieved by administering about 284 mg of inclisiran (equivalent to about 300 mg of inclisiran sodium), e.g. subcutaneously, and wherein the method comprises:
(a) a loading phase comprising administering inclisiran as two doses separated by a time interval of about 90 days (3 months), followed by
(b) a maintenance phase comprising administering one or more doses of inclisiran, wherein each dose is separated by a time interval of about 180 days (6 months).
   The time intervals can be varied to account for any missed doses, as described further below.

Administration of the RNAi agent also reduces the level of PCSK9 in the subject. In some embodiments, administration of the RNAi agent may reduce the level of PCSK9, as compared to a baseline PCSK9 level or as compared to the level of PCSK9 before administration of the RNAi agent, by greater than about 25%, or by greater than about 30%, or by greater than about 35%, or by greater than about 40%, or by greater than about 45%, or by greater than about 50%, or by greater than about 55%, or by greater than about 60%, or by greater than about 65%, or by greater than about 70%, or by greater than about 75%, or by greater than about 80%, or by greater than about 85%, or by greater than about 90%, or by greater than about 95%.

In some embodiments, the reduction in the PCSK9 level after the RNAi agent is administered may be maintained for, at, or through about 30 days or more, 60 days or more, 90 days or more, 180 days or more, 1 year or more, 2 years or more, 3 years or more, 4 years or more, 5 years or more, or 6 years or more.

In an exemplary embodiment, these reductions in the level of PCSK9 can be achieved by administering about 284 mg of inclisiran (equivalent to about 300 mg of inclisiran sodium), e.g. subcutaneously, and wherein the method comprises:
(a) a loading phase comprising administering inclisiran as two doses separated by a time interval of about 90 days (3 months), followed by
(b) a maintenance phase comprising administering one or more doses of inclisiran, wherein each dose is separated by a time interval of about 180 days (6 months).

### Subjects for Treatment

The methods of the invention are useful in reducing the risk of a MACE in subjects that have not experienced a major atherosclerotic cardiovascular disease (ASCVD) event.

### Subjects at an Increased Risk for a First MACE

These methods are particularly useful in reducing the risk of a MACE in subjects that are at an increased risk for a first MACE.

The increased risk for a first MACE is defined as any one of the following:
a. Evidence of atherosclerotic CAD on CT or invasive coronary angiogram defined as a coronary artery stenosis ≥20% but <50% in the left main coronary artery or a stenosis ≥20% but <70% in any major epicardial coronary artery;
b. Coronary artery calcium (CAC) score obtained by CT-scan ≥100 Agatston units;
c. High 10-year ASCVD risk ≥20% (assessed with the Pooled Cohort Equation);
d. Intermediate 10-year ASCVD risk 7.5%—<20% (assessed with the Pooled Cohort Equation) with at least two of the following risk-enhancing factors:
   i. A first degree relative with history of premature ASCVD (males, age <55 years; females, age <65 years)
   ii. History of premature menopause before age 40
   iii. History of maternal pregnancy complications (defined as a history of either preeclampsia, gestational hypertension, or gestational diabetes)
   iv. South Asian ancestry
   v. Chronic inflammatory disorders (defined as either rheumatoid arthritis, systemic lupus erythematosus, psoriasis covering ≥10% of body surface area, Crohn's disease, ulcerative colitis, or HIV/AIDS)
   vi. High-sensitivity C-reactive protein (hsCRP) ≥2 mg/L, and, in absence of any underlying acute or inflammatory conditions (including any of the chronic inflammatory disorders listed under criteria d. v., above)
   vii. Lipoprotein (a) ≥50 mg/dL or 125 nmol/L
   viii. Estimated glomerular filtration rate (eGFR)<60 mL/min/1.73 m²
   ix. Ankle branchial index (ABI)<0.9 with no symptoms of intermittent claudication; or
   x. Metabolic syndrome.

The skilled person is able to determine whether a subject is at increased risk of a first MACE, as defined by the criteria above.

For example, Achenbach and Raggi (2010) reviews exemplary methods of determining evidence of atherosclerotic CAD on CT or invasive coronary angiogram, and determing a CAC score obtained by CT-scan.

The Pooled Cohort Equation for assessing ASCVD risk is explained in further detail below.

Criteria for diagnosis of metabolic syndrome are also explained in further detail below.

The levels of high-sensitivity C-reactive protein (hsCRP), Lipoprotein (a), estimated glomerular filtration rate (eGFR), and LDL-C in a subject can be determined by the skilled person using routine tests, for example commercially available immunoassays or commercially available tests (e.g. lipid panels) to measure levels of these substances in a subject (e.g. in the serum obtained from a subject).

The skilled person is also able to, for example, calculate the ankle branchial index (ABI) in a subject by measuring their blood pressure in the artery of the ankle and arm of the subject.

In some embodiments, the subject has not experienced a MACE at any time prior to administration of the RNAi agent.

In some embodiments, the subject has LDL-C level ≥70 mg/dL (≥1.81 mmol/L) but <190 mg/dL (<4.91 mmol/L).

In some embodiments, the subject is a mammal, for example a primate. In preferred embodiments of all aspects of the invention, the subject is a human. In some embodiments, the subject is referred to as the patient.

In some embodiments, the subject is an adult (e.g. a human adult), for example greater than or equal to 40 years of age, and optionally less than 80 years of age.

### Background Lipid-Lowering Therapy, Additional PCSK9-Targeted Therapy, and Other Therapy

In some embodiments, the subject is being treated or has been treated with a background lipid-lowering therapy (LLT). In other embodiments, the subject has not been or is not being treated with a background lipid-lowering therapy (LLT). The subject may remain on the background LLT during the time in which the RNAi agent is administered to said subject, as described herein. The background LLT may be a statin and/or ezetimibe. The background statin LLT may be moderate-intensity or high-intensity.

In some embodiments, the subject has not been treated with an additional PCSK9-targeted therapy prior to administration of the RNAi agent. In other embodiments, the subject has been treated with an additional PCSK9-targeted therapy prior to administration of the RNAi agent. Exemplary additional PCSK9-targeted therapies include PCSK9-blocking monoclonal antibodies (mAbs) which lower LDL-C levels, such as Alirocumab or Evolocumab, and consequently decrease levels of LDL-C.

The RNAi agent (e.g. inclisiran) can be administered immediately after the last dose of a monoclonal antibody PCSK9 inhibitor. Preferably, the RNAi agent (e.g. inclisiran) is administered within 2 weeks of the last dose of a monoclonal antibody PCSK9 inhibitor.

In some embodiments, the subject has not been treated with lomitapide and/or mipomersen.

### Pooled Cohort Equation

The pooled cohort equations (PCE) were introduced in the 2013 ACC/AHA guidelines on CV risk as a sex- and race-specific tool for estimating 10-year absolute rates of ASCVD events in a primary prevention population (Goff et al 2014). These equations were derived from extensive data from several large, racially and geographically diverse, modern NHLBI-sponsored cohort studies, including the ARIC (Atherosclerosis Risk in Communities) study, Cardiovascular Health Study, and the CARDIA (Coronary Artery Risk Development in Young Adults) study, combined with applicable data from the Framingham Original and Offspring Study cohorts. The risk estimates are based on a combination of established CV risk factors examined prospectively in specific cohorts selected for derivation of the PCE. If required, the ASCVD risk score will be calculated using the PCE equation on the eCRF for inclusion criteria assessment.

Variables included in the PCE are:
Current age
Sex (male, female)
Race (White, African American, Other)
Smoking status (current, former, never)
Systolic blood pressure (in mmHg)
Hypertension treatment status (yes, no)
History of diabetes (yes, no)
Total and HDL cholesterol levels (in mg/dL or mmol/L)

Further information about ASCVD risk tool is described in Lloyd-Jones et al, 2018. The tool estimates the 10-year ASCVD risk for adults between 40-79 years of age.

### Criteria for Clinical Diagnosis of Metabolic Syndrome

Metabolic Syndrome is a Risk-Enhancing Factor for ASCVD. The Diagnosis of Metabolic Syndrome Will be Made by the Presence of any 3 of the Following 5 Risk Factors:

### Risk Factors to Support the Diagnosis of Metabolic Syndrome

| Measure | Categorical Cut Points |
|---|---|
| Elevated waist circumference¹ | ≥102 cm (40.1 in) in males |
| | ≥88 cm (34.6 in) in females |
| Elevated triglycerides (drug | ≥150 mg/dL (1.69 mmol/L) |
| treatment for elevated | |
| triglycerides is an alternative | |
| indicator)² | |
| Reduced HDL-C (drug treatment | <40 mg/dL (1.03 mmol/L) |
| for reduced HDL-C is an | in males |
| alternative indicator)² | <50 mg/dL (1.29 mmol/L) |
| | in females |
| Hypertension (antihypertensive | SBP ≥130 and/or DBP ≥85 |
| drug treatment in a patient with | mmHg |
| a history of hypertension is an | |
| alternative indicator) | |
| Elevated fasting plasma glucose | ≥100 mg/dL (5.55 mmol/L) |
| (drug treatment for elevated | |
| glucose is an alternative indicator) | |

SBP = systolic blood pressure;
DBP = diastolic blood pressure
¹Lower cut points should be used for Asian subjects: ≥90 cm (35.4 in) in males and ≥80 cm (31.5 in) in females.
²The most commonly used drug treatments for elevated triglycerides and reduced HDL-C are fibrates, nicotinic acid, high doses of n-3 polyunsaturated fatty acids (PUFAS) and icosapent ethyl.
A patient taking one of these drug treatments will be presumed to have high triglycerides and low HDL-C.

Adapted from: Grundy et al 2019 (Table S2 in the Web Supplement) and Grundy et al 2004

These risk factors can be measured or determined in a subject by methods known to a person skilled in the art.

### Primary Prevention

As mentioned above, the methods of the invention are useful in reducing the risk of a MACE in subjects that have not experienced a major atherosclerotic cardiovascular disease (ASCVD) event. This is also known to the skilled person as ‘primary prevention’.

The major ASCVD event can be defined as any one of the following:
a. Acute coronary syndrome (ACS) at any time prior to administration of the RNAi agent, for example ACS within about 12 months prior to administration of the RNAi agent;
b. Myocardial infarction at any time prior to administration of the RNAi agent;
c. Ischemic stroke at any time prior to administration of the RNAi agent; or
d. Symptomatic peripheral artery disease (PAD), for example as evidenced by either intermittent claudication, previous revascularization, or amputation due to atherosclerotic disease, at any time prior to administration of the RNAi agent.

In exemplary embodiments, the subject has not experienced a major ASCVD within about 12 months prior to administration of the RNAi agent.

Additional factors may indicate that the subject is not suitable for the methods of the invention. The following embodiments define subjects that are particularly suitable for the methods of the invention with reference to these factors.

In some embodiments, the subject does not have a history of ischemia-driven revascularization in a coronary or extracoronary arterial bed prior to administration of the RNAi agent.

In some embodiments, the subject has an absence of coronary atherosclerosis on a CT angiogram or an invasive coronary angiogram in the 2 years prior to administration of the RNAi agent.

In some embodiments, the subject has not had a coronary artery calcium (CAC) score of 0 obtained in the 2 years prior to administration of the RNAi agent.

In some embodiments, the subject does not have active liver disease or hepatic dysfunction, for example identified as having aspartate aminotransferase (AST) or alanine aminotransferase (ALT) greater than or equal to 3 times the upper limit of normal (ULN).

### Subgroups of Subjects

The methods of the invention may be particularly suitable or efficacious for certain subgroups of subjects.

In some embodiments, the subject may have ASCVD, ASCVD risk equivalent, an elevated risk for cardiovascular disease (CVD), heterozygous familial hypercholesterolemia, or homozygous familial hypercholesterolemia, is in need of lowering low density lipoprotein cholesterol (LDL-C), or otherwise has a disorder that would benefit from a reduction in LDL-C, or a combination thereof.

In certain embodiments, a subject may be “ASCVD risk equivalent” if they have one or more of the following: symptomatic atherosclerosis, Type 2 diabetes, familial hypercholesterolemia, including subjects whose 10-year risk of a cardiovascular event assessed by Framingham Risk Score (score >20%) or equivalent has a target LDL-C of <100 mg/dl. In some embodiments, the subject may have heterozygous familial hypercholesterolemia (HeFH).

The Framingham Risk Score is a well known method to the skilled person, used to estimate 1-year cardiovascular disease risk, which takes into account factors such as age, HDL-C levels, total cholesterol levels, systolic blood pressure, and smoking and diabetes history, which is described further in D'agostino et al. 2008.

The Pooled Cohort Equation can also be used to estimate ASCVD risk.

In some embodiments, the subject may be less than 65 years of age, 65 to 74 years of age, or at least 75 years of age. The subject may be male or female.

In some embodiments, the subject has a baseline body mass index (BMI) of less than 30, 30 to less than 35, or at least 35 kg/m².

In some embodiments, the subject has or had diabetes mellitus.

In some embodiments, the subject is a current or past smoker.

In some embodiments, the subject has or had hypertension.

In some embodiments, the subject has baseline LDL-C levels of less than 100 mg/dL, or at least 100 mg/dL.

In some embodiments, the subject has baseline triglyceride levels of less than 150, 150 to less than 200, or at least 200 mg/dL.

In some embodiments, the subject has baseline high sensitivity C reactive protein (hs-CRP) levels of less than 2 or at least 2 mg/dL.

In some embodiments, the subject is being treated with baseline statin at high intensity or moderate intensity, or is not being treated with statin.

In some embodiments, the subject may have a potential to develop cardiac health issues, which may include: having acute coronary syndromes; having a history of myocardial infarction, stable or unstable angina, coronary or other arterial revascularization, stroke, transient ischemic attack, or peripheral arterial disease of atherosclerotic origin; being male; having a family history of heart disease, ASCVD, or ASCVD risk equivalent; having a smoking habit; being physically inactive; having high blood pressure; having high blood cholesterol; having diabetes or prediabetes; being overweight or obese; having a history of preeclampsia during pregnancy; having uncontrolled stress and/or anger; being post-menopausal; having an unhealthy diet, e.g., a diet high in salt, saturated fat, trans fat, cholesterol, and/or refined sugars; being age 55 or older; having sleep apnea; having anemia; or a combination thereof. Therefore, the method can be directed to, for example, subjects that are young but have a familial history of heart disease, or subjects that smoke but do not have high blood pressure.

### Other Interventions

The subjects to be treated in the methods of the invention may be treated with background LLT throughout the time in which the RNAi agent is administered. Additionally, additional risk-based cardiovascular disease (CVD) preventative interventions may be delivered to the subjects, such as education on healthy diet, exercise, and weight control, or other lifestyle changes. The subjects may be on a maximally tolerated statin therapy. The maximum dosages for statins are known in the art.

Such healthy diets are known in the art, such as diets designed to improve lipid levels. For instance, a diet designed to improve lipid levels may comprise eating lean cuts of meat; removing fat from meats; not eating fried foods or high-fat sauces; not eating egg yolks; using low-fat dairy products such as skim milk or 1% milk, low-fat frozen yogurt, low-fat ice cream and low-fat cheeses; and eating foods that are sources of fiber, such as fruits and vegetables.

The subjects may require additional lowering of LDL-C levels.

### RNAi Agents

The RNAi agent used in the methods of the invention is a double stranded RNAi agent. The RNAi agent is capable of inhibiting the expression of Proprotein convertase subtilisin kexin 9 (PCSK9) in a cell.

In some aspects of the invention, the RNAi agent is a double-stranded ribonucleic acid comprising a sense strand and an antisense strand that form a double-stranded region, wherein the antisense strand comprises the nucleotide sequence of SEQ ID NO: 1 and the sense strand comprises the nucleotide sequence of SEQ ID NO: 2. In some embodiments, the antisense strand comprises the nucleotide sequence of SEQ ID NO: 3 and the sense strand comprises the nucleotide sequence of SEQ ID NO: 4.

In some aspects of the invention, the RNAi agent comprises:
an antisense strand comprising the nucleotide sequence 5′-asCfsaAfAfAfgCfaAfaAfcAfgGfuCfuagsasa-3′ (SEQ ID NO: 3), and a sense strand comprising the nucleotide sequence 5′-csusagacCfuGfudTuugcuuuugu-3′ (SEQ ID NO: 4),
wherein a, g, c, and u are 2′-O-methyl (2′-OMe) modified A, G, C, and U nucleotides, respectively; Af, Gf, Cf and Uf are 2′-fluoro A, G, C and U modified nucleotides, respectively; dT is a deoxy-thymine nucleotide and s is a phosphorothioate linkage; and
wherein the sense strand is conjugated to at least one ligand.

In some aspects of the invention, the RNAi agent comprises:
an antisense strand consisting of the nucleotide sequence 5′-asCfsaAfAfAfgCfaAfaAfcAfgGfuCfuagsasa-3′ (SEQ ID NO: 3), and
a sense strand consisting of the nucleotide sequence 5′-csusagacCfuGfudTuugcuuuugu-3′ (SEQ ID NO: 4),
wherein a, g, c, and u are 2′-O-methyl (2′-OMe) modified A, G, C, and U nucleotides, respectively; Af, Gf, Cf and Uf are 2′-fluoro A, G, C and U modified nucleotides, respectively; dT is a deoxy-thymine nucleotide and s is a phosphorothioate linkage; and
wherein the sense strand is conjugated to at least one ligand.

In some embodiments, the ligand comprises the following structure:

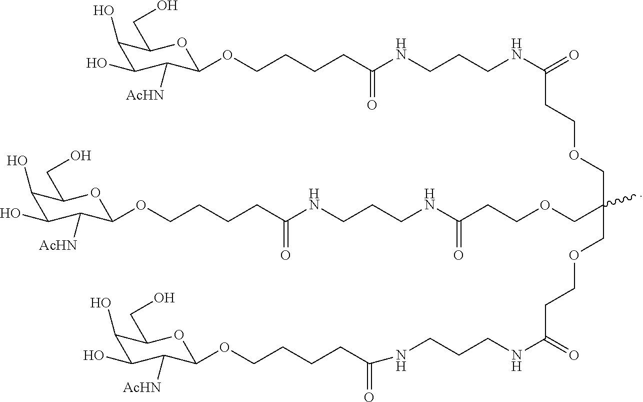 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

This structure represents a triantennary N-Acetylgalactosamine (GalNAc), which facilitates uptake of the RNAi agent by hepatocytes. In hepatocytes, the RNAi agent utilizes the RNA interference mechanism and directs catalytic breakdown of the mRNA for PCSK9. This increases LDL-C receptor recycling and expression on the hepatocyte cell surface, which increases LDL-C uptake and lowers LDL-C levels in the circulation.

In further embodiments, the ligand is conjugated to the 3′ end of the sense strand.

The RNAi agent can be conjugated to the ligand as shown in the following schematic:

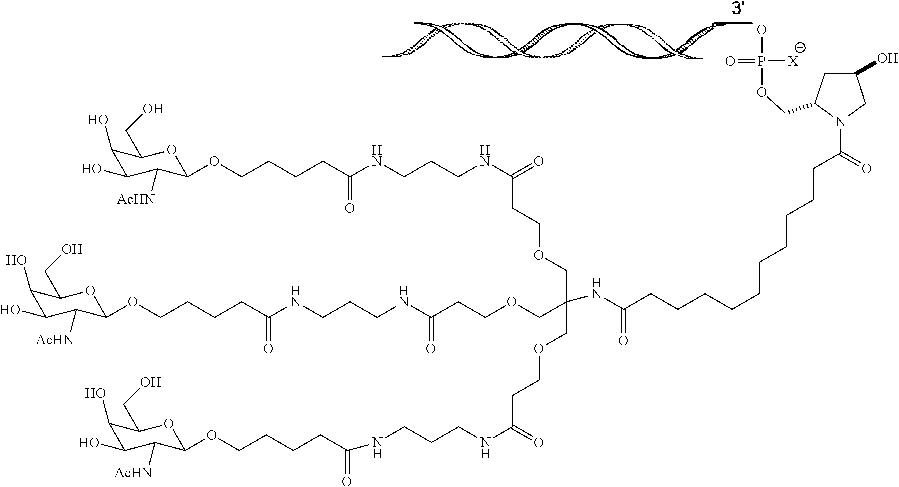

wherein X is O or S. In preferred embodiments, X is O.

Preferably, the RNAi agent is inclisiran. Inclisiran is the preferred RNAi agent for all aspects of the invention. Inclisiran is a double-stranded RNAi agent which is a small interfering RNA (siRNA) directed to PCSK9 (proprotein convertase subtilisin kexin type 9) mRNA. Inclisiran is conjugated at the 3′ end of the sense strand with triantennary N-Acetylgalactosamine (GalNAc) to facilitate uptake by hepatocytes, as described above. With one exception, the 2′ ribose moieties of inclisiran are present as 2′-F or 2′-OMe ribonucleotide. In addition, six of the terminal phosphodiester backbones are present as phosphorothioate linkages as indicated below.

Inclisiran is depicted on pages 504-506 of Recommended INN: List 76 from the WHO Drug Information, Vol. 30, No. 3, 2016, which is incorporated by reference.

The chemical name of inclisiran is:
duplex of [(2S,4R)-1-{1-[(2-acetamido-2-deoxy-β-D-galactopyranosyl)oxy]-16,16-bis({3-[(3-{5-[(2-acetamido-2-deoxy-β-D-galactopyranosyl)oxy]pentanamido}propyl)amino]-3-oxopropoxy}methyl)-5,11,18-trioxo-14-oxa-6,10,17-triazanonacosan-29-oyl}-4-hydroxypyrrolidin-2-yl]methyl hydrogen
all-P-ambo-2′-O-methyl-P-thiocytidylyl-
(3′→5′)-2′-O-methyl-P-thiouridylyl-
(3′→5′)-2′-O-methyladenylyl-
(3′→5′)-2′-O-methylguanylyl-
(3′→5′)-2′-O-methyladenylyl-
(3′→5′)-2′-O-methylcytidylyl-
(3′→5′)-2′-deoxy-2′-fluorocytidylyl-
(3′→5′)-2′-O-methyluridylyl-
(3′→5′)-2′-deoxy-2′-fluoroguanylyl-
(3′→5′)-2′-O-methyluridylyl-
(3′→5′)-thymidylyl-
(3′→5′)-2′-O-methyluridylyl-
(3′→5′)-2′-O-methyluridylyl-
(3′→5′)-2′-O-methylguanylyl-
(3′→5′)-2′-O-methylcytidylyl-
(3′→5′)-2′-O-methyluridylyl-
(3′→5′)-2′-O-methyluridylyl-
(3′→5′)-2′-O-methyluridylyl-
(3′→5′)-2′-O-methyluridylyl-
(3′→5′)-2′-O-methylguanylyl-
(3′→5′)-2′-O-methyl-3′-uridylate
and
all-P-ambo-2′-O-methyl-P-thioadenylyl-
(5′→3′)-2′-O-methyl-P-thioadenylyl-
(5′→3′)-2′-O-methylguanylyl-
(5′→3′)-2′-O-methyladenylyl-
(5′→3′)-2′-O-methyluridylyl-
(5′→3′)-2′-deoxy-2′-fluorocytidylyl-
(5′→3′)-2′-O-methyluridylyl-
(5′→3′)-2′-deoxy-2′-fluoroguanylyl-
(5′→3′)-2′-O-methylguanylyl-
(5′→3′)-2′-deoxy-2′-fluoroadenylyl-
(5′→3′)-2′-O-methylcytidylyl-
(5′→3′)-2′-deoxy-2′-fluoroadenylyl-
(5′→3′)-2′-O-methyladenylyl-
(5′→3′)-2′-deoxy-2′-fluoroadenylyl-
(5′→3′)-2′-O-methyladenylyl-
(5′→3′)-2′-deoxy-2′-fluorocytidylyl-
(5′→3′)-2′-O-methylguanylyl-
(5′→3′)-2′-deoxy-2′-fluoroadenylyl-
(5′→3′)-2′-deoxy-2′-fluoroadenylyl-
(5′→3′)-2′-deoxy-2′-fluoroadenylyl-
(5′→3′)-2′-O-methyl-P-thioadenylyl-
(5′→3′)-2′-deoxy-2′-fluoro-P-thiocytidylyl-
(5′→3′)-2′-O-methyladenosine.

In the chemical name of inclisiran (shown above), the annotation (3′→5′) or (5′→3′) refers to the direction of the internucleoside linkages (i.e. the direction of each of the inter-connecting phospho link, for example phosphodiester or phosphorothioate), as is IUPAC convention for nucleic acids, and not to the end of the strand (e.g. the antisense or sense strand) that has the third or fifth carbon in the sugar-ring at its terminus.

The molecular formula of inclisiran is C₅₂₉H₇₀₇F₁₂N₁₇₆O₃₁₆P₄₃S₆.

The structure of inclisiran is shown in the following diagram:

```
        (3′-5′) C=U=A-G-A-C-C-U-G-U-T-U-U-G-C-U-U-U-U-G-U-R1
                        • • • • • • • • • • • • • • • • • • • • •
(5′-3′) A=A=G-A-U-C-U-G-G-A-C-A-A-A-A-C-G-A-A-A-A=C=A
```

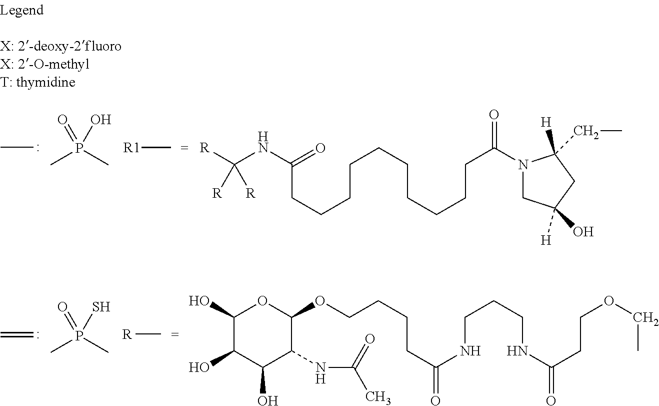

The following illustration is an alternative representation of the structure of inclisiran:

```
Sense Strand
5′ 3′
                Cms-Ums-Am-Gm-Am-Cm-Cf-Um-Gf-Um-dT-Um-Um-Gm-Cm-Um-Um-Um-Um-Gm-Um-L96
                • • • • • • • • • • • • • • • • • • • • •
Ams-Ams-Gm-Am-Um-Cf-Um-Gf-Gm-Af-Cm-Af-Am-Af-Am-Cf-Gm-Af-Af-Af-Ams-Cfs-Am
Antisense Strand
3′ 5′
```

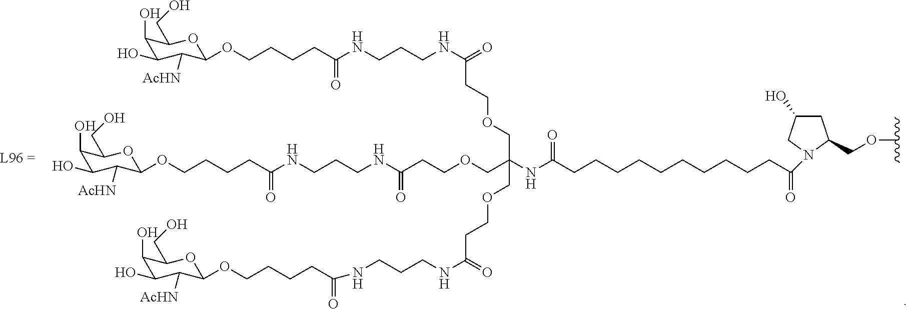 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

Abbreviations: Af=adenine 2′-F ribonucleotide; Cf=cytosine 2′-F ribonucleotide; Gf=guanine 2′-F ribonucleotide; Am=adenine 2′-OMe ribonucleotide; Cm=cytosine 2′-OMe ribonucleotide; Gm=guanine 2′-OMe ribonucleotide; Um=uracil 2′-OMe ribonucleotide; s is a phosphorothioate linkage.

### Pharmaceutical Compositions and Administration

### Salt Forms

Each dose of the administered RNAi agent may comprise the RNAi agent in the salt form.

As used herein, the terms “salt” or “salts” refer to an acid addition or base addition salt of an RNAi agent described herein. “Salts” include in particular “pharmaceutically acceptable salts”. The term “pharmaceutically acceptable salts” refers to salts that retain the biological effectiveness and properties of the RNAi agents described herein and, which typically are not biologically or otherwise undesirable. In many cases, the RNAi agents described herein are capable of forming base salts by virtue of the presence of acidic protons, for example as found in carboxylic or phosphoric acids or other groups similar thereto.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, RNAi agents described herein are in sodium, potassium, ammonium, calcium, magnesium, iron, silver, zinc, copper, isopropylamine, benzathine, cholinate, diethanolamine, diethylamine, lysine, meglumine, piperazine or tromethamine salt form.

In some embodiments, the RNAi agent is present in the dose to be administered as a salt, for example a sodium salt or a potassium salt. Sodium salts are preferred salt forms of the RNAi agent, but other pharmaceutically acceptable salts can be used, for example potassium or ammonium salts. Preferably, the RNAi agent is an inclisiran salt (for example inclisiran sodium, inclisiran potassium, or inclisiran ammonium). More preferably, the RNAi agent is inclisiran sodium (also known as inclisiran sodium salt), but other pharmaceutically acceptable salts can also be used.

The molecular formula of inclisiran sodium is C₅₂₉H₆₆₄F₁₂N₁₇₆Na₄₃O₃₁₆P₄₃S₆ and its molecular weight is 17,284.72 g/mol. It has the following structural formula:

```
Sense Strand 21 Na
5′ 3′
                Cms-Ums-Am-Gm-Am-Cm-Cf-Um-Gf-Um-dT-Um-Um-Gm-Cm-Um-Um-Um-Um-Gm-Um-L96
                • • • • • • • • • • • • • • • • • • • •
Ams-Ams-Gm-Am-Um-Cf-Um-Gf-Gm-Af-Cm-Af-Am-Af-Am-Cf-Gm-Af-Af-Af-Ams-Cfs-Am
3′ 5′
Antisense Strand 22 Na
Inclisiran Sodium
```

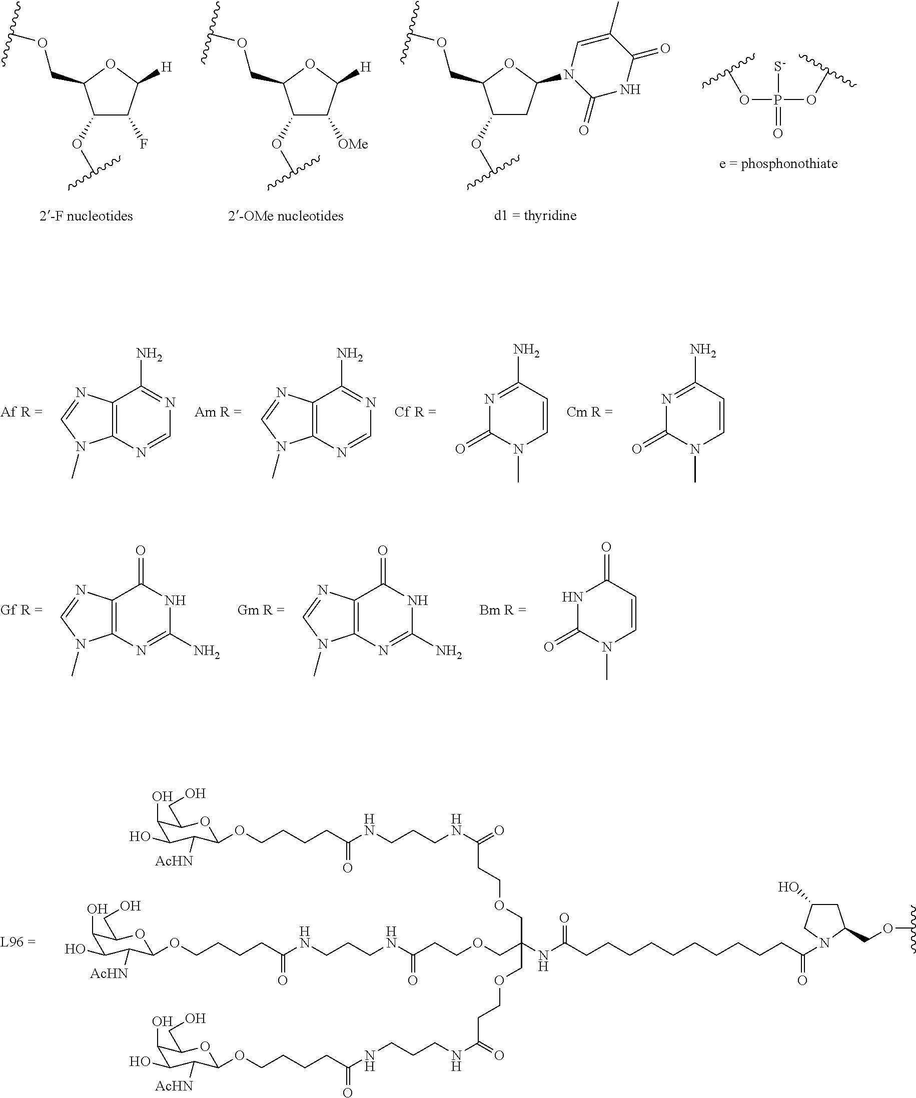

Abbreviations: Af=adenine 2′-F ribonucleotide; Cf=cytosine 2′-F ribonucleotide; Gf=guanine 2′-F ribonucleotide; Am=adenine 2′-OMe ribonucleotide; Cm=cytosine 2′-OMe ribonucleotide; Gm=guanine 2′-OMe ribonucleotide; Um=uracil 2′-OMe ribonucleotide; s is a phosphorothioate linkage.

### RNAi Agents for Use and Pharmaceutical Compositions for Use

The present invention also provides an RNAi agent for use in any of the methods of the invention. Also provided are pharmaceutical compositions comprising an RNAi agent for use in any of the methods of the invention.

These pharmaceutical compositions may comprise, in addition to the RNAi agent or salt thereof, a pharmaceutically acceptable excipient, carrier, buffer, stabilizer, or other material swell known to those skilled in the art. Such materials are non-toxic and do not interfere with the efficacy of the RNAi agent. In preferred embodiments, the pharmaceutical composition for use comprises water for injection. In further preferred embodiments, the pharmaceutical composition for use comprises water for injection, and sodium hydroxide and/or phosphoric acid.

### Dosages and Dosage Regimens

### Doses

The methods of the invention comprise administering the RNAi agent as one or more doses. The RNAi agent may be provided in a therapeutically effective amount or a prophylactically effective amount. In all aspects of the invention, the person skilled in the art will recognize that administration of the RNAi agent may comprise administering a therapeutically effective amount or a prophylactically effective amount of the RNAi agent to the subject in each dose.

The reference to a ‘therapeutically effective amount’ or ‘prophylactically effective amount’ refers to administering an appropriate amount of the RNAi agent to achieve the effect of reducing the risk of a major adverse cardiovascular event (MACE) in a subject that has not experienced a major atherosclerotic cardiovascular disease (ASCVD) event, in accordance with the methods of the invention. It is also apparent to the skilled person that the terms ‘therapeutically effective amount’ or ‘prophylactically effective amount’ may refer to the effect of reducing the risk of an event as described in further aspects of the invention described herein, in a subject that has not experienced a major atherosclerotic cardiovascular disease (ASCVD) event, for example reducing the risk of first or recurrent non-fatal MI, non-fatal ischemic stroke, urgent revascularization, hospitalizations for unstable angina, heart failure hospitalization, or major adverse limb events (MALE), etc.

Each dose administered to the subject may comprise between about 100 mg to about 500 mg of inclisiran salt. In some embodiments, each dose administered to the subject comprises about 100 mg, 200 mg, 300 mg, 400 mg, or 500 mg of inclisiran salt. Preferably, the inclisiran salt is inclisiran sodium.

Each dose administered to the subject may comprise between about 94 mg to about 473 mg of inclisiran. In particular embodiments, each dose administered to the subject comprises about 95 mg of inclisiran (equivalent to about 100 mg of inclisiran sodium), 189 mg of inclisiran (equivalent to about 200 mg of inclisiran sodium), 284 mg of inclisiran (equivalent to about 300 mg of inclisiran sodium), 388 mg of inclisiran (equivalent to about 400 mg of inclisiran sodium), or about 473 mg of inclisiran (equivalent to about 500 mg of inclisiran sodium). Inclisiran may be comprised in these doses in other salt forms as the equivalent amount of inclisiran salt, such as those described above.

In some embodiments, each dose administered to the subject comprises about 284 mg of inclisiran (or the equivalent amount of inclisiran salt). Preferably, each dose comprises about 284 mg of inclisiran (equivalent to about 300 mg of inclisiran sodium). This amount of inclisiran is considered to be an exemplary therapeutically effective amount or an exemplary prophylactically effective amount.

In preferred embodiments, a dose of about 284 mg of inclisiran (equivalent to about 300 mg of inclisiran sodium) is administered as a single subcutaneous injection initially, again at 3 months, and then every 6 months.

### Loading Phase and Maintenance Phase

In some embodiments, the administration of the RNAi agent comprises a loading phase followed by a maintenance phase.

The loading phase may comprise administering the RNAi agent as one or more doses, for example administering the RNAi agent as at least two doses each separated by a time interval. Preferably, the loading phase comprises administering the RNAi agent as two doses separated by a time interval.

The administration of each dose in the loading phase may be separated by a time interval of about 30 days (1 month) to 270 days (9 months). In some embodiments, the administration of each dose in the loading phase may be separated by a time interval of about 90 days (3 months) to 180 days (6 months). For example, the administration of each dose in the loading phase is separated by a time interval of about 30 days (1 month), about 60 days (2 months), about 90 days (3 months), about 120 days (4 months), about 150 days (5 months), or about 180 days (6 months). Preferably, the administration of each dose in the loading phase is separated by a time interval of about 90 days (3 months).

In some embodiments, the administration of the maintenance phase comprises administering the RNAi agent as one or more doses, each separated by a time interval. In exemplary embodiments, the time interval between the administration of each dose is about 90 days (3 months) to about 270 days (9 months). Preferably, the time interval is about 180 days (6 months). In further preferred embodiments, the time intervals between the administration of each dose in the maintenance phase are of about equal duration—for example, the time interval between the first and second doses of the maintenance phase may be about 180 days (6 months), and the time interval between the second and third doses of the maintenance phase may be about 180 days (6 months).

In some embodiments, the time interval between the administration of each dose is about 90 days (3 months) to about 270 days (9 months). For example, this time interval may be between the administration of each dose in the maintenance phase, or two separate doses from the loading phase and the maintenance phase, respectively. In some embodiments, the first dose of the maintenance phase is administered about 180 days (6 months) after the last dose of the loading phase.

The duration of the maintenance phase may be at least 1 year. Preferably, the duration of the maintenance phase is at least 2 years, at least 3 years, at least 4 years, at least 5 years, or at least 6 years. The methods of the invention are particularly useful in reducing the risk of a MACE in a subject that has not experienced a major atherosclerotic cardiovascular disease (ASCVD) event when the RNAi agent is administered during a long maintenance phase (e.g. at least 4 years, at least 5 years, or at least 6 years), due to the effective and sustained reduction in LDL-C levels in a subject. Administration of 284 mg of inclisiran in the form of 300 mg of inclisiran sodium to a subject initially, again at 3 months, and then every 6 months has been shown in the ORION-3 open-label extension trial to provide effective and sustained reductions in the subjects' low-density lipoprotein cholesterol (LDL-C) over 4 years of treatment. LDL-C level reduction was sustained over the four-year study period: subjects treated with inclisiran achieved an average 47.5% reduction in LDL-C from baseline (Day 1 of ORION-1) to Day 210 (95% CI:−50.69,−44.27) and a time-averaged reduction in LDL-C of 44.2% over the four years through twice-yearly dosing.

Accordingly, the RNAi agent may be administered to the subject initially, again at 3 months, and then every 6 months. In some embodiments, the administration of the RNAi agent comprises:
(a) a loading phase comprising administering the RNAi agent as two doses separated by a time interval of about 90 days (3 months), followed by
(b) a maintenance phase comprising administering one or more doses of the RNAi agent, each separated by a time interval of about 180 days (6 months).
The time intervals can be varied to account for any missed doses, as described further below.

### Missed Doses

In the methods of the invention, the time points at which the RNAi agent is intended or expected to be administered according to a particular (original) dosing schedule (e.g. initially, again at 3 months, and then every 6 months) are also referred to as planned doses.

If any planned dose is missed by less than 90 days (3 months), the missed planned dose of the RNAi agent is administered immediately. In this situation, the next dose of the RNAi agent is typically administered according to the original dosing schedule.

If any planned dose is missed by more than 90 days (3 months), the dosing schedule is restarted by administering the RNAi agent in a new loading phase comprising a first dose and a second dose about 90 days (3 months) after the first dose, followed by a maintenance phase comprising administering one dose about every 180 days (6 months) (i.e., the first dose of the maintenance phase is administered about 180 days (6 months) after the second dose of the loading phase).

### Administration Routes

In view of the mechanism of action of the RNAi agents of the invention, subcutaneous administration of the RNAi agent is particularly suitable. Preferably, the RNAi agent is administered subcutaneously (s.c.), preferably by injection into the abdomen, upper arm, or thigh. Administration by subcutaneous injection into the abdomen is particularly preferred.

The RNAi agent for subcutaneous administration may be comprised in a pharmaceutical composition. The pharmaceutical composition may be formulated based on the mode of delivery, which is readily achievable by the person skilled in the art.

The RNAi agent may be administered in a volume of about of 1.5 mL of a sterile aqueous solution. Preferably, the sterile aqueous solution has a pH of about 7.0. The sterile aqueous solution may contain sodium hydroxide and/or phosphoric acid for pH adjustment, to a target pH of about 7.0. The sterile aqueous solution comprising the RNAi agent is typically contained in a prefilled syringe for subcutaneous administration. The RNAi agent may be in water for injection, in the pharmaceutical composition.

The RNAi agent may also be administered intravenously (i.v.). In some embodiments, the RNAi agent is administered to the subject subcutaneously and intravenously, for example the RNAi agent is administered subcutaneously for one dose and intravenously for another dose. The person skilled in the art is able to prepare suitable aqueous solutions for subcutaneous and/or intravenous administration (e.g. the RNAi agent may be comprised in a sterile aqueous solution of about pH 7.0), and administer the RNAi agent subcutaneously and/or intravenously. Examples of routine techniques and protocols can be found in Remington: The Science and Practice of Pharmacy, 23rd Edition, 2020.

In particular embodiments wherein the RNAi agent is administered intravenously, each dose administered to the subject may comprise between about 100 mg to about 500 mg of inclisiran salt. In some embodiments, each dose administered to the subject comprises about 100 mg, 200 mg, 300 mg, 400 mg, or 500 mg of inclisiran salt. Preferably, the inclisiran salt is inclisiran sodium. Each dose administered to the subject may comprise between about 94 mg to about 473 mg of inclisiran. In particular embodiments, each dose administered to the subject comprises about 95 mg of inclisiran (equivalent to about 100 mg of inclisiran sodium), 189 mg of inclisiran (equivalent to about 200 mg of inclisiran sodium), 284 mg of inclisiran (equivalent to about 300 mg of inclisiran sodium), 388 mg of inclisiran (equivalent to about 400 mg of inclisiran sodium), or about 473 mg of inclisiran (equivalent to about 500 mg of inclisiran sodium). Inclisiran may be comprised in these doses in other salt forms as the equivalent amount of inclisiran salt, such as those described above.

In particular embodiments wherein the RNAi agent is administered intravenously, each dose administered to the subject comprises about 284 mg of inclisiran (or the equivalent amount of inclisiran salt). Preferably, each dose comprises about 284 mg of inclisiran (equivalent to about 300 mg of inclisiran sodium).In some embodiments, the method comprises administering the RNAi agent intravenously in the loading phase and administering the RNAi agent subcutaneously in the maintenance phase. In other embodiments, the method comprises administering the RNAi agent intravenously for the first dose of the loading phase, administering the RNAi agent subcutaneously for each subsequent dose in the loading phase, and administering the RNAi agent subcutaneously for each dose in the maintenance phase.

In further embodiments, the method comprises administering the RNAi agent intravenously for the first dose of the loading phase, and administering the RNAi agent subcutaneously for each subsequent dose. In embodiments where the method comprises administering the RNAi agent intravenously for the first dose of the loading phase, and administering the RNAi agent subcutaneously for each subsequent dose, each dose in the loading phase may be separated by a time interval of about 30 days (1 month) to 270 days (9 months). In some of these embodiments, each dose in the loading phase may be separated by a time interval of about 90 days (3 months) to 180 days (6 months). For example, each dose in the loading phase is separated by a time interval of about 30 days (1 month), about 60 days (2 months), about 90 days (3 months), about 120 days (4 months), about 150 days (5 months), or about 180 days (6 months). Preferably, each dose in the loading phase is separated by a time interval of about 90 days (3 months).

The RNAi agent may be administered in an outpatient setting. The RNAi agent is typically administered by a healthcare professional.

### Exemplary Embodiments

The following exemplary embodiments of the invention are provided:

Embodiment 1. A method of reducing the risk of a major adverse cardiovascular event (MACE) in a subject that has not experienced a major atherosclerotic cardiovascular disease (ASCVD) event,
wherein the MACE is cardiovascular death, non-fatal myocardial infarction (MI), non-fatal ischemic stroke, or urgent coronary revascularization,
wherein the method comprises administering inclisiran subcutaneously to the subject, and the administration comprises:
(a) a loading phase comprising administering inclisiran as two doses separated by a time interval of about 90 days (3 months), followed by
(b) a maintenance phase comprising administering one or more doses of inclisiran, wherein each dose is separated by a time interval of about 180 days (6 months), and
each dose comprises about 284 mg of inclisiran (equivalent to about 300 mg of inclisiran sodium).

Embodiment 2. A method of reducing the risk of a major adverse cardiovascular event (MACE) in a subject that has not experienced a major atherosclerotic cardiovascular disease (ASCVD) event,
wherein the MACE is cardiovascular death, non-fatal myocardial infarction (MI), non-fatal ischemic stroke, or urgent coronary revascularization,
wherein the major ASCVD event is defined as any one of the following:
   a. Acute coronary syndrome (ACS) at any time prior to administration of the RNAi agent, for example ACS within about 12 months prior to administration of the RNAi agent;
   b. Myocardial infarction at any time prior to administration of the RNAi agent;
   c. Ischemic stroke at any time prior to administration of the RNAi agent; or
   d. Symptomatic peripheral artery disease (PAD), for example as evidenced by either intermittent claudication, previous revascularization, or amputation due to atherosclerotic disease, at any time prior to administration of the RNAi agent,
wherein the method comprises administering inclisiran subcutaneously to the subject, and the administration comprises:
(a) a loading phase comprising administering inclisiran as two doses separated by a time interval of about 90 days (3 months), followed by
(b) a maintenance phase comprising administering one or more doses of inclisiran, wherein each dose is separated by a time interval of about 180 days (6 months), and
each dose comprises about 284 mg of inclisiran (equivalent to about 300 mg of inclisiran sodium).

### General

The practice of the present invention will employ, unless otherwise indicated, conventional methods of chemistry, biochemistry, molecular biology, immunology and pharmacology, within the skill of those in the art.

The term “comprising” encompasses “including” as well as “consisting”, e.g., a composition “comprising” X may consist exclusively of X or may include something additional, e.g., X+Y.

The term “about” in relation to a numerical value x is optional and means, for example, x±10%, unless indicated otherwise.

The term “baseline” may refer to a condition without treatment, such as before the treatment was first administered or initiated.

As used in this specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.All technical and scientific terms used herein have the same meaning as commonly understood by one of ordinary skill in the art to which this application belongs and as commonly used in the art to which this application belongs. The publications and other reference materials referenced herein to describe the background of the invention and to provide additional detail regarding its practice are hereby incorporated by reference.

### EXAMPLES

The following examples are included for illustrative purposes only and are not intended to limit the scope of the invention.

### Example 1. Trial to Evaluate the Effect of Inclisiran on Preventing Major Adverse Cardiovascular Events in High-Risk Primary Prevention Patients

A randomized, double-blind, placebo-controlled multicenter study to evaluate the effect of inclisiran on preventing major adverse cardiovascular events in high-risk primary prevention patients will be conducted.

This is a Phase III study designed to test the hypothesis that treatment with inclisiran in the form of inclisiran sodium 300 milligram (mg) subcutaneously (s.c.) administered initially (on Day 1), Day 90, and every 6 months thereafter in patients at high cardiovascular (CV) risk without a prior major atherosclerotic cardiovascular disease (ASCVD) event will significantly reduce the risk of 4-Point-Major Adverse Cardiovascular Events (4P-MACE), defined as a composite of CV death, non-fatal myocardial infarction (MI), non-fatal ischemic stroke, and urgent coronary revascularization, compared to placebo.

### Methods

The purpose of this study is to evaluate inclisiran sodium 300 mg s.c. (subcutaneous) (equivalent to 284 mg inclisiran) compared to placebo on reducing the risk of 4P-MACE in adult patients at high risk for their first major adverse cardiovascular event. Randomized participants will receive study medication (inclisiran or placebo), administered s.c. on Day 1, Day 90, then every 6 months thereafter. The overall trial duration is expected to be approximately 6.3 years, and the study will continue until at least 853 participants have experienced a Clinical Endpoint Committee (CEC)-confirmed primary 4P-MACE (composite of CV death, non-fatal (MI), non-fatal ischemic stroke, and urgent coronary revascularization), and at least 638 participants have experienced a CEC-confirmed 3P-MACE (composite of CV death, non-fatal MI, and non-fatal ischemic stroke) across both treatment arms, and all participants have had a minimum of 3 years of follow-up (during the double-blind period).

It is expected that inclisiran will have at least a 20% reduction on average in the hazard rate in 4P-MACE endpoint compared to placebo.

It is also expected that inclisiran will have at least a 20% reduction on average in the hazard rate in 3P-MACE endpoint compared to placebo.

### Appropriateness of Efficacy Assessments

4P-MACE, 3P-MACE, and cardiovascular death are well-defined and standard clinical assessments in clinical trials aimed at demonstrating CV risk reduction. Assessments of events and laboratory tests related to the secondary endpoints are in line with the expected efficacy of inclisiran.

Study objectives and related endpoints

| Objective(s) | Endpoint(s) |
|---|---|
| Primary objective(s) | Endpoint(s) for primary objective(s) |
| To demonstrate the superiority of inclisiran compared to | Time to the first occurrence of 4P-MACE |
| placebo in reducing the risk of 4P-MACE (composite of | |
| CV death, non-fatal MI, non-fatal ischemic stroke, and | |
| urgent coronary revascularization) | |
| Secondary objective(s) | Endpoint(s) for secondary objective(s) |
| To demonstrate the superiority of inclisiran compared | Time to the first occurrence of 3P-MACE |
| to placebo in reducing the risk of 3P-MACE | |
| (composite of CV death, non-fatal MI, and non-fatal | |
| ischemic stroke) | |
| To demonstrate the superiority of inclisiran compared | Times to the occurrences of CV death, non- |
| to placebo in reducing the rate of total 4P-MACE | fatal MI, non-fatal ischemic stroke, and |
| (composite of CV death, non-fatal MI, non-fatal | urgent coronary revascularization (first and |
| ischemic stroke, and urgent coronary | recurrent) |
| revascularization) | |
| To demonstrate the superiority of inclisiran compared | Times to the occurrences of CV death, non- |
| to placebo in reducing the rate of total 3P-MACE | fatal MI, and non-fatal ischemic stroke (first |
| (composite of CV death, non-fatal MI, and non-fatal | and recurrent) |
| ischemic stroke) | |
| To demonstrate the superiority of inclisiran compared | Time to CV death |
| to placebo in reducing the risk of CV death | |
| To demonstrate the superiority of inclisiran compared | Time to all-cause mortality |
| to placebo in reducing the risk of all-cause mortality | |
| To evaluate the safety and tolerability of inclisiran | Number of participants with SAEs |
| compared to placebo | Number of participants with Aes leading to |
| | study drug discontinuation |
| Exploratory objective(s) | Endpoint(s) for exploratory objective(s) |
| To evaluate inclisiran compared to placebo in reducing | Time to the first occurrence of any CHD |
| the risk of any coronary heart disease (CHD) event | event (death from CHD, non-fatal MI, |
| (death from CHD, non-fatal MI, unstable angina | unstable angina requiring hospitalization, or |
| requiring hospitalization, and urgent coronary | urgent coronary revascularization) |
| revascularization) | |
| To evaluate inclisiran compared to placebo in reducing | Time to the first occurrence of fatal or non- |
| the risk of fatal or non-fatal MI | fatal MI |
| To evaluate inclisiran compared to placebo in reducing | Time to the first occurrence of fatal or non- |
| the risk of fatal or non-fatal ischemic stroke | fatal ischemic stroke |
| To evaluate inclisiran compared to placebo in reducing | Time to CV death |
| the risk of CV death, while borrowing information from | |
| VICTORION-2 PREVENT study (CKJX839B12302) | |
| To evaluate inclisiran compared to placebo in reducing | Time to the first occurrence of all-cause |
| the risk of the composite endpoint of all-cause | mortality, non-fatal MI, or non-fatal |
| mortality, non-fatal MI, and non-fatal ischemic stroke | ischemic stroke |
| To evaluate inclisiran compared to placebo in reducing | Time to the first occurrence of all-cause |
| the risk of the composite endpoint of all-cause | mortality, non-fatal MI, non-fatal ischemic |
| mortality, non-fatal MI, non-fatal ischemic stroke, and | stroke, or urgent coronary revascularization |
| urgent coronary revascularization | |
| To evaluate inclisiran compared with placebo in | Time to the first occurrence of MALE |
| reducing the risk of major adverse limb events (MALE) | |
| (including acute lower limb ischemia, lower limb | |
| amputation due to ischemia, and urgent lower limb | |
| revascularization for ischemia) | |
| To evaluate inclisiran compared to placebo in reducing | Times to the occurrences of all-cause |
| the rate of all-cause revascularizations (coronary, | revascularization procedures (first and |
| carotid, peripheral) | recurrent) |
| To evaluate inclisiran compared to placebo in reducing | Times to the occurrences of coronary |
| the rate of total coronary events (CHD death, MI, | events (first and recurrent) |
| hospitalizations for unstable angina, and | |
| urgent/elective revascularizations) | |
| To evaluate inclisiran compared to placebo in reducing | Times to the occurrences of all-cause CV |
| the rate of all-cause CV hospitalizations, including | hospitalizations (first and recurrent) |
| hospitalizations for MI, and hospitalizations for | |
| unstable angina | |
| To evaluate inclisiran compared to placebo in reducing | Times to the occurrences of venous |
| the rate of venous thromboembolisms resulting in | thromboembolisms resulting in |
| hospitalization or death | hospitalizations or death (first and |
| | recurrent) |
| To evaluate health economic benefits of inclisiran | Times to the occurrences of CV events |
| compared to placebo | including but not limited to non-fatal MI, |
| | non-fatal ischemic stroke, urgent |
| | revascularization, hospitalizations for |
| | unstable angina, heart failure |
| | hospitalization, or MALE (first and |
| | recurrent) |
| To evaluate inclisiran compared to placebo in reducing | Percentage change in LDL-C and other |
| LDL-C and other lipid parameters (total cholesterol, | lipid parameters from baseline at time |
| high-density lipoprotein cholesterol (HDL-C), very low- | points specified in Schedule of Activities |
| density lipoprotein cholesterol (VLDL-C), non-high- | Absolute change in LDL-C and other lipid |
| density lipoprotein cholesterol (non-HDL-C), | parameters from baseline at time points |
| triglycerides and lipoprotein(a) [Lp(a)]) from baseline | specified in Schedule of Activities |

AE = adverse event;
MI = myocardial infarction;
3P-MACE = 3-point-major adverse cardiovascular events;
4P-MACE = 4-point-major adverse cardiovascular events;
SAE = serious adverse event

### Primary Endpoint

The primary endpoint is time to first occurrence of 4P-MACE, a composite of CV death, non-fatal MI, non-fatal ischemic stroke, and urgent coronary revascularization.

### Secondary Endpoints

The secondary endpoints considered for efficacy are:
Time to the first occurrence of 3P-MACE (CV death, non-fatal MI, and non-fatal ischemic stroke)
Times to the occurrences of 4P-MACE (First and recurrent CV death, non-fatal MI, non-fatal ischemic stroke, and urgent coronary revascularization)
Times to the occurrences of 3P-MACE (First and recurrent CV death, non-fatal MI, and non-fatal ischemic stroke)
Time to CV death
Time to all-cause mortality

### Exploratory Endpoints

The other cardiovascular (CV) endpoints assessed will be as follows:
CV death
Non-fatal MI
Non-fatal ischemic stroke
Urgent coronary revascularization
Composite of all-cause mortality, non-fatal MI, and non-fatal ischemic stroke
Composite of all-cause mortality, non-fatal MI, non-fatal ischemic stroke, and urgent coronary revascularization
Composite of coronary events (death from CHD, MI, unstable angina requiring hospitalization, and urgent/elective revascularization)
Major adverse limb events (MALE)
All-cause urgent revascularization
All-cause CV hospitalizations, including hospitalizations for MI and hospitalizations for unstable angina
Venous thromboembolisms resulting in hospitalization or death
Total CV events (first and recurrent non-fatal MI, non-fatal ischemic stroke, hospitalizations for unstable angina, hospitalization for heart failure or MALE)

### Other Efficacy Assessments

Other efficacy assessments will include: total cholesterol, LDL-C, HDL-C, VLDL-C, non-HDL-C, Lp(a), and triglycerides. Participants must be fasting for 10 hours prior to visits with lipid/lipoprotein assessments. A lipid panel will be assessed at every study visit after randomization (Day 1), following the Assessment Schedule.

### Patient Population for Study

The study will involve 14,000 patients at high CV risk without a prior major ASCVD event, with LDL-C ≥70 mg/dL (1.81 mmol/L) but <190 mg/dL (4.91 mmol/L).

### Inclusion Criteria

Participants eligible for inclusion in this study must meet all of the following criteria:
1. Signed informed consent must be obtained prior to participation in the study
2. Male or female ≥40 but <80 years of age
3. At an increased risk for a first MACE (i.e., no prior major ASCVD event), defined as any one of the following:
   a. Evidence of atherosclerotic CAD on CT or invasive coronary angiogram defined as a coronary artery stenosis ≥20% but <50% in the left main coronary artery or a stenosis ≥20% but <70% in any major epicardial coronary artery
   b. Coronary artery calcium (CAC) score obtained by CT-scan ≥100 Agatston units—determined at any time before the screening visit
   c. High 10-year ASCVD risk ≥20% (assessed with the Pooled Cohort Equation)
   d. Intermediate 10-year ASCVD risk 7.5%—<20% (assessed with the Pooled Cohort Equation) with at least 2 of the following risk-enhancing factors:
      i. A first degree relative with history of premature ASCVD (males, age <55 years; females, age <65 years)
      ii. History of premature menopause before age 40
      iii. History of maternal pregnancy complications (defined as a history of either preeclampsia, gestational hypertension, or gestational diabetes)
      iv. South Asian ancestry
      v. Chronic inflammatory disorders (defined as either rheumatoid arthritis, systemic lupus erythematosus, psoriasis covering ≥10% of body surface area, Crohn's disease, ulcerative colitis, or HIV/AIDS)
      vi. High-sensitivity C-reactive protein (hsCRP) ≥2 mg/L—documented in the 12 months preceding the screening visit, and, in absence of any underlying acute or inflammatory conditions (including any of the chronic inflammatory disorders listed under criteria 3 d. v. above)
      vii. Lipoprotein (a) ≥50 mg/dL or 125 nmol/L—determined at any time before the screening visit
      viii. Estimated glomerular filtration rate (eGFR)<60 mL/min/1.73 m²—documented between the screening and randomization visits
      ix. Ankle branchial index (ABI)<0.9 with no symptoms of intermittent claudication
      x. Metabolic syndrome: increased waist circumference, elevated triglycerides, elevated blood pressure, elevated glucose, and low HDL-C are factors—a tally of 3 or more, documented between the screening and randomization visits
4. If on a background LLT, the dose should be stable for at least 4 weeks prior to the screening visit and the participant should be willing to remain on this background therapy for the entire duration of the study.
5. LDL-C ≥70 mg/dL (≥1.81 mmol/L) but <190 mg/dL (<4.91 mmol/L) at the screening visit

### Exclusion Criteria

Patients are excluded from the study if they meet any of the following exclusion criteria:
1. History of major ASCVD event defined as any one of the following:
   a. Acute coronary syndrome (ACS) in the 12 months prior to randomization, or
   b. Prior myocardial infarction at any time prior to randomization, or
   c. Prior ischemic stroke at any time prior to randomization, or
   d. Symptomatic peripheral artery disease (PAD) as evidenced by either intermittent claudication, previous revascularization, or amputation due to atherosclerotic disease, at any time prior to randomization
2. History of, or planned, ischemia-driven revascularization in a coronary or extracoronary arterial bed prior to randomization
3. Absence of coronary atherosclerosis on a CT angiogram or an invasive coronary angiogram in the 2 years prior to randomization
4. Coronary artery calcium (CAC) score of 0 obtained in the 2 years prior to randomization.
5. Active liver disease or hepatic dysfunction, defined as AST or ALT ≥3×ULN from central laboratory test at screening visit
6. Current or planned renal dialysis or transplantation
7. Previous (within 90 days prior to the screening visit), current, or planned treatment with a mAb directed toward PCSK9 (e.g., evolocumab, alirocumab)
8. Previous exposure to inclisiran or any other non-mAb PCSK9-targeted therapy, either as an investigational or marketed drug within 2 years prior to randomization
9. Severe concomitant non-cardiovascular disease that is expected to reduce life expectancy to less than 5 years
10. Use of other investigational drugs within 5 half-lives of enrollment, or within 30 days (e.g., for small molecules), or until the expected pharmacodynamic effect has returned to baseline (e.g., for biologics), whichever is longer
11. History of hypersensitivity to any of the study drugs or its excipients or to drugs of similar chemical classes
12. Any surgical or medical condition, which in the opinion of the Investigator, may place the participant at higher risk from his/her participation in the study, or is likely to prevent the participant from complying with the requirements of the study or completing the study
13. Unwillingness or inability (e.g., physical or cognitive) to comply with study procedures (including adherence to study visits, fasting blood draws and compliance with study treatment regimens), and medication administration (injections) and schedule
14. Pregnant or nursing (lactating) women
15. Women of child-bearing potential, defined as all women physiologically capable of becoming pregnant, unless they are using effective methods of contraception while taking study treatment.
16. Persons directly involved in the execution of this protocol including investigational study staff and their family members

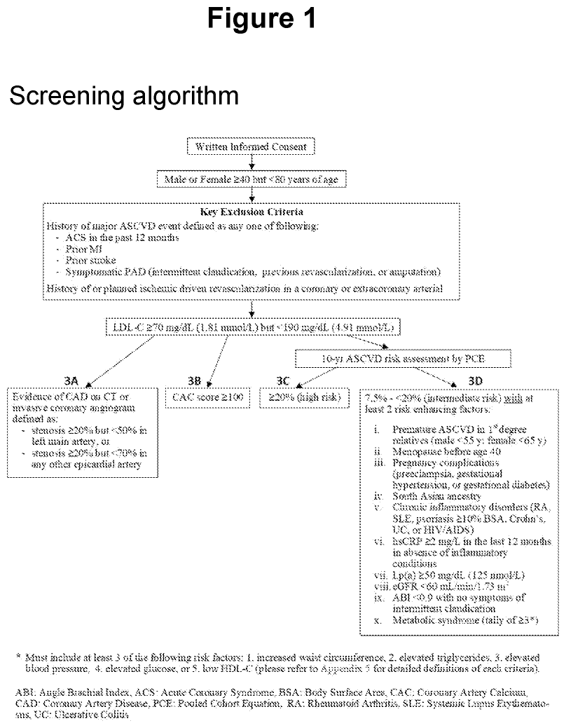

A summary of the screening algorithm for the screening strategy is shown in FIG. 1.

### Study Treatments

### Study Design

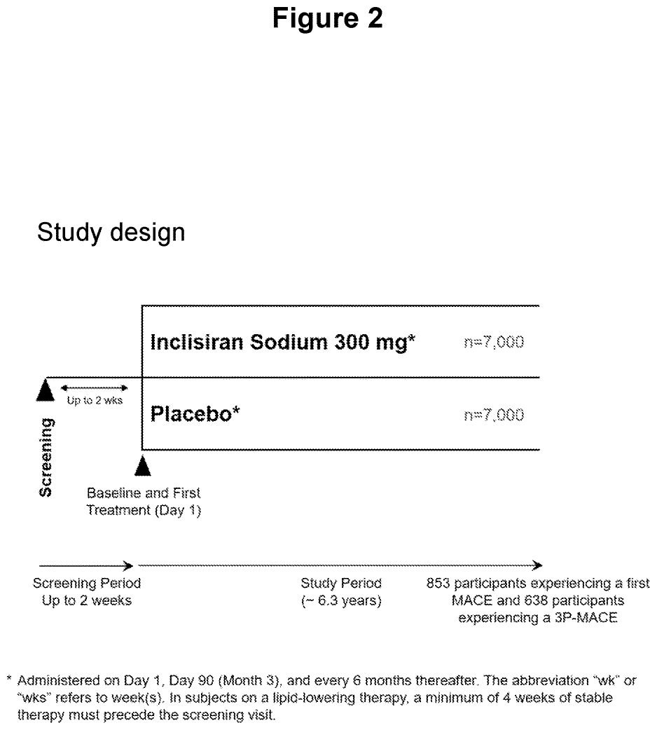

A diagram of the study design is shown in FIG. 2.

### Investigational and Control Drug

| Investigational/ | Treatment Form or | | |
|---|---|---|---|
| Control Drug | Pharmaceutical | Route of | |
| (Name and Strength) | Dosage Form | Administration | Presentation |
| Inclisiran sodium 300 | Solution for | Subcutaneous | Solution for injection |
| mg (equivalent to 284 | injection | injection | in pre-filled syringe |
| mg inclisiran) in 1.5 mL | | | |
| Placebo in 1.5 mL | Solution for | Subcutaneous | Solution for injection |
| | injection | injection | in pre-filled syringe |

### Additional Study Treatments

No other treatment beyond investigational drug and control drug are included in this trial.

### Treatment Arms/Group

Participants will be assigned at the Day 1 (Baseline) visit to one of the following two treatment arms/groups in a 1:1 ratio:
Inclisiran sodium 300 mg (equivalent to about 284 mg of inclisiran) s.c.
Matching placebo s.c.

Each participant will receive one s.c. injection of blinded inclisiran or placebo on Day 1, a second injection of blinded inclisiran or placebo on Month 3, and subsequently an injection of blinded inclisiran or placebo every 6 months thereafter.

### Treatment Duration

The study duration is expected to be approximately 6.3 years. However, as this is an event-driven trial, the treatment duration will vary depending on when a participant enters the study. The study will complete when at least 853 participants have experienced a Clinical Endpoint Committee (CEC)-confirmed primary 4P-MACE and at least 638 participants have experienced a CEC-confirmed 3P-MACE across both treatment arms, and all randomized participants completed 3 years of follow-up. Participants may be discontinued from the study drug earlier due to safety reasons and/or at the discretion of the Investigator or the participants. If this occurs, they will continue to be followed in the study unless they withdraw their informed consent.

### Instruction for Prescribing and Taking Study Treatment

Investigational and control drugs will not be dispensed to the participants. Study drug administration will be performed by the Investigator or a qualified healthcare professional.

Participants will be administered a single s.c. injection of 300 mg inclisiran sodium or placebo at pre-defined time points as described in the Schedule of Activities. Injections will be administered after all other study assessments have been completed for the visit.

The preferred site of injection is the abdomen. Alternative injection sites include the upper arm or thigh. Injections should not be given into areas of active skin disease or injury such as sunburns, skin rashes, inflammation, or skin infections.

### Scientific Rationale for Study Design

This study is aimed to evaluate the efficacy and safety of inclisiran sodium 300 mg on reducing the risk of 4P-MACE in participants at high CV risk, but without a prior major ASCVD event (i.e., primary prevention), with elevated LDL-C levels despite being on guideline-recommended LLT. This study design is the most appropriate trial design to answer the above clinical question of interest. The LDL-C entry criteria of ≥70 mg/dL (1.81 mmol/L) is based on several lipid treatment guidelines in patients at high ASCVD risk (Grundy et al 2019, Mach et al 2020).

### Justification for Dose

The established dose regimen for inclisiran sodium for adults is 300 mg administered s.c. at Day 1, Month 3 (Day 90), and every 6 months thereafter. This dose and dose regimen were extensively studied in the Phase III clinical program with 1,833 patients exposed to inclisiran and will be used in the present study to provide maximum efficacy with an acceptable safety profile. Furthermore, this dose and dose regimen has been approved in the United States, Europe, and other countries around the world for the reduction of LDL-C (Leqvio USPI, Leqvio SmPC).

This dose and dose regimen were selected based on data from the Phase II dose-finding study (ORION-1). ORION-1 tested different doses of inclisiran sodium up to 500 mg, as single or multiple doses given 90 days apart (Ray et al 2019). This study found that the regimen comprising two induction doses of 300 mg inclisiran sodium yielded the greatest reduction in LDL-C from baseline to Day 180 (52.6%). This dose and regimen also yielded the greatest reduction in PCSK9 from baseline to Day 180 (69.1%). Further providing evidence for the 300 mg dose, a Phase I study (ALN-PCSSC-001) studied doses up to 900 mg and did not find a clinically significant additional benefit beyond 300 mg in that study (Fitzgerald et al 2017). Furthermore, a 300 mg dose of inclisiran sodium has been studied in participants with mild, moderate and severe renal impairment and mild and moderate hepatic impairment, with no clinically meaningful differences in LDL-C and PCSK9 lowering compared to that observed in other participant populations, and with no dose adjustment required Therefore, this dosing regimen is efficacious in patients including those with renal and hepatic impairment.

Three large pivotal Phase III clinical trials (ORION-9, -10, -11) (Wright et al 2021) in adults with ASCVD, ASCVD risk equivalents, and HeFH demonstrated that the dose regimen used in the present study (300 mg inclisiran sodium s.c. on Day 1, Month 3 (Day 90), and every 6 months thereafter) resulted in placebo-adjusted percentage reductions in LDL-C from baseline at Day 510 of 48% to 52%, with time-adjusted average reductions of 44% to 54% sustained over 18 months (Raal et al 2020, Ray et al 2020). This dose and regimen have been shown to be well tolerated, with a safety profile similar to placebo, with the exception of more injection site reactions associated with inclisiran compared to placebo.

The duration of treatment will last for the full course of the study, which is expected to be approximately 6.3 years. The study will complete when the target number of events and minimum follow-up has occurred.

### Rationale for Choice of Background Therapy

Participants should be on a standard of care background LLT according to local practice guidelines. While practice guidelines vary across countries, some general principles are used in the present protocol and are aligned with the most recent US-based guideline on the management of blood cholesterol (Grundy et al 2019).

More specifically, patients entering the study with evidence of CAD (inclusion criteria 3a), a CAC score ≥100 Agatston units (inclusion criteria 3b) or diabetes mellitus are required to be on at least a moderate-intensity statin therapy for a minimum of 4 weeks prior to the screening visit.

In line with the most recent US-based guidelines (Grundy et al 2019) and for maximal ASCVD risk reduction, a high-intensity statin therapy is strongly recommended for the following categories of patients:

Patients entering the trial with a high 10-year ASCVD risk ≥20% (inclusion criteria 3c);

Patients with an intermediate 10-year ASCVD risk (≥7.5-<20%) plus 2 risk-enhancing factors which, per guidelines, reclassifies them in the high 10-year ASCVD risk category (inclusion criteria 3d); and

Regardless of other inclusion criteria, patients with diabetes mellitus and multiple ASCVD risk factors (as assessed by clinical judgment).

These recommendations of high-intensity statin therapy can be adapted, if applicable, to local practice guidelines as well as clinical judgment.

In all instances, the dose(s) of any LLT should be stable for at least 4 weeks prior to the screening visit and cannot include a PCSK9-targeted treatment, and the participant should be willing to remain on this background LLT for the entire duration of the study.

During the conduct of the study, the Sponsor will ensure that approximately 70% of randomized participants enter the trial with a statin in their background therapy.

### Rationale for Choice of Control Drugs (Comparator/Placebo)

This trial is placebo-controlled, on top of standard of care, to provide robust evidence on the effects of inclisiran on clinical events, safety, and tolerability. The use of placebo as a comparator is justified because all randomized participants will be treated according to local practice guidelines prior to entering the study, and for the entire duration of the study. All participants will receive standard of care including, but not limited to dietary advice, antihypertensives, antihyperglycemic agents (if applicable), and antiplatelet therapies provided by the Investigator, as clinically appropriate.

### Concomitant Therapy

### Concomitant Lipid-Lowering Therapy

Participants are required to maintain their background LLT unchanged during the study (i.e., no medication or dose changes).

Changes to the background LLT are only allowed under the following circumstances:
Interruption, dose reduction or discontinuation of the background LLT can be done in case of AEs requiring such changes.
   Dose Increase or Adding Another Background LLT is Allowed if the LDL-C Increases Above a Pre-Defined Threshold. Prohibited Medication

Use of the treatments displayed in the below table are not allowed after screening.

Prohibited medication

| | Prohibition | |
|---|---|---|
| Medication | period | Action taken |
| Monoclonal antibodies | Full study | Discontinue study treatment, but the participant should |
| (mAbs) directed towards | period | return for all subsequent study visits as indicated in the |
| PCSK9 such as | | Assessment Schedule. In the exceptional circumstance |
| evolocumab and | | that 1 or 2 doses were administered since the prior visit |
| alirocumab | | and outside of the study protocol, Novartis must be |
| | | contacted for additional guidance. |
| Any other non-mAb | Full study | Discontinue study treatment, but the participant should |
| PCSK9-targeted therapy | period | return for all subsequent study visits as indicated in the |
| other than study drug | | Assessment Schedule |
| (including commercially | | |
| available inclisiran) | | |
| Lomitapide | Full study | Discontinue study treatment, but the participant should |
| | period | return for all subsequent study visits as indicated in the |
| | | Assessment Schedule |
| Mipomersen | Full study | Discontinue study treatment, but the participant should |
| | period | return for all subsequent study visits as indicated in the |
| | | Assessment Schedule |
| Any other investigational | Full study | Discontinue study treatment, but the participant should |
| treatments | period | return for all subsequent study visits as indicated in the |
| | | Assessment Schedule |

mAb = monoclonal antibody

| | Period | | | | | | | | | | |
|---|---|---|---|---|---|---|---|---|---|---|---|
| | Screening | Double-Blind Treatment Period¹ | | | | | | | | | |
| | Visit Name | | | | | | | | | | |
| | | Baseline | | | | | | | | | |
| | Screening | (V1) | V2 | V3 | V4 | V5 | V6 | V7 | V8 | V9 | V10 |
| | Days | | | | | | | | | | |
| | −14 to −1 | 1 | 90 | 270 | 450 | 630 | 810 | 990 | 1170 | 1350 | 1530 |
| | Months | | | | | | | | | | |
| | −1 | 0 | 3 | 9 | 15 | 21 | 27 | 33 | 39 | 45 | 51 |
| Informed consent | X | | | | | | | | | | |
| Inclusion/exclusion criteria | X | X | | | | | | | | | |
| Demography | X | | | | | | | | | | |
| Smoking history | X | | | | | | | | | | |
| Medical history/current | X | | | | | | | | | | |
| medical conditions | | | | | | | | | | | |
| Prior or concomitant | X | X | X | X | X | X | X | X | X | X | X |
| medications | | | | | | | | | | | |
| Lipid lowering medications | X | X | X | X | X | X | X | X | X | X | X |
| (type/dose) | | | | | | | | | | | |
| Surgical/medical procedures | X | X | X | X | X | X | X | X | X | X | X |
| Physical examination | S³ | | | | | | | | | | |
| Body Height | X | | | | | | | | | | |
| Body Weight | X | | X | X | X | X | X | X | X | X | X |
| Waist circumference | X | | | | | | | | | | |
| Vital signs⁴ | X | X | X | X | X | X | X | X | X | X | X |
| Lifestyle instructions | | X | X | X | X | X | X | X | X | X | X |
| Pregnancy test⁵ | X | | | | | | | | | | |
| Fasting lipid profile⁶ | X | X | X | X | X | X | X | X | X | X | X |
| Fasting Lp(a) | | X | X | | X | | | | | | |
| Broad clinical chemistry⁷ | X | | | | | | | | | | |
| Liver function tests⁸ | | X | X | | X | | X | | X | | X |
| Hematology⁹ | X | | | | | | | | | | |
| Urinalysis¹⁰ | X | | | | | | | | | | |
| hsCRP | | X | | | | | | | | | |
| Genetic consent | X | | | | | | | | | | |
| Genetic testing¹¹ | | X | | | | | | | | | |
| Exploratory biomarkers¹² | | X | X | | X | | | | | | |
| AE/SAE assessment¹³ | X | X | X | X | X | X | X | X | X | X | X |
| Enter patient visit into IRT | X | X | X | X | X | X | X | X | X | X | X |
| Randomization | | X | | | | | | | | | |
| Study drug administration | | X | X | X | X | X | X | X | X | X | X |
| Endpoint assessment | | | X | X | X | X | X | X | X | X | X |

| | Period | | | | | |
|---|---|---|---|---|---|---|
| | | | | | End of | Safety |
| | Double-Blind Treatment Period¹ | | | | Study | follow up |
| | Visit Name | | | | | |
| | | | | | | Safety |
| | | | | | | follow- |
| | V11 | V12 | V13 | V14 | EOS² | up call |
| | Days | | | | | |
| | | | | | | EOS +30 |
| | 1710 | 1890 | 2070 | 2250 | EOS | days |
| | Months | | | | | |
| | 57 | 63 | 69 | 75 | | |
| Informed consent | | | | | | |
| Inclusion/exclusion criteria | | | | | | |
| Demography | | | | | | |
| Smoking history | | | | | | |
| Medical history/current | | | | | | |
| medical conditions | | | | | | |
| Prior or concomitant | X | X | X | X | X | |
| medications | | | | | | |
| Lipid lowering medications | X | X | X | X | X | |
| (type/dose) | | | | | | |
| Surgical/medical procedures | X | X | X | X | X | |
| Physical examination | | | | | S³ | |
| Body Height | | | | | | |
| Body Weight | X | X | X | X | X | |
| Waist circumference | | | | | | |
| Vital signs⁴ | X | X | X | X | X | |
| Lifestyle instructions | X | X | X | X | X | |
| Pregnancy test⁵ | | | | | S³ | |
| Fasting lipid profile⁶ | X | X | X | X | X | |
| Fasting Lp(a) | | | | | | |
| Broad clinical chemistry⁷ | | | | | | |
| Liver function tests⁸ | | X | | X | X | |
| Hematology⁹ | | | | | | |
| Urinalysis¹⁰ | | | | | | |
| hsCRP | | | | | | |
| Genetic consent | | | | | | |
| Genetic testing¹¹ | | | | | | |
| Exploratory biomarkers¹² | | | | | | |
| AE/SAE assessment¹³ | X | X | X | X | X | S³ |
| Enter patient visit into IRT | X | X | X | X | X | |
| Randomization | | | | | | |
| Study drug administration | X | X | X | X | | |
| Endpoint assessment | X | X | X | X | X | |

X Assessment to be recorded in the clinical database or received electronically from a vendor
S Assessment to be recorded in the source documentation only
¹This is an event driven trial. The follow-up period may be longer or shorter than 6 years. If the participant continues beyond Month 75 (6.25 years), each subsequent visit should be scheduled at 6-month increments (e.g., the next visit would be at Month 81, followed by Month 87) until the trial is completed. Each of these subsequent visits will follow the Assessment Schedule for the visit 12 months prior (e.g., Month 81 would follow Month 69 assessments, and Month 87 would follow Month 75 assessments), including study drug administration and IRT call.
²The End of Study Visit will be the visit following the last study drug injection after the trial completes, an early discontinuation for any reason, or after a study end date has been determined by the DMC.
³Recorded in source documentation only
⁴Blood pressure and pulse will be taken 3 times in sitting position
⁵Only in women of childbearing potential. Serum test at screening will be performed at central laboratory. Urine test at EOS will be performed locally via urine dipstick
⁶Total cholesterol, LDL-C, non-HDL-C, HDL-C, VLDL-C, triglycerides. Participants must be fasting for 10 hours prior to visit.
⁷AST, ALT, ALP, total bilirubin (direct and indirect bilirubin if total bilirubin >2x ULN), creatinine, eGFR, fasting plasma glucose, HbA1c, creatine kinase, chloride, sodium, potassium. Participants must be fasting for 10 hours prior to visit.
⁸Performed among a subset of patients. AST, ALT, ALP, total bilirubin (direct and indirect bilirubin if total bilirubin >2x ULN).
⁹Hematocrit, hemoglobin, mean cell hemoglobin (MCH), mean corpuscular hemoglobin concentration (MCHC), mean corpuscular volume (MCV), platelets, red blood cells (RBC), white blood cells (WBC), differential (% of basophils, eosinophils, lymphocytes, monocytes, neutrophils).
¹⁰Specific gravity, pH, glucose, protein (total), ketones, bilirubin, urobilinogen, nitrite, hemoglobin (blood), leukocyte esterase.
¹¹Blood samples to be collected and stored for genetic testing. This sample will require a separate Informed Consent.
¹²Performed among a subset of patients.
¹³Only AEs leading to discontinuation, SAEs, and AEs of special interest will be collected.

### Analysis

### Primary Endpoint(s) Estimand(s) Analysis

The primary aim of the study is to demonstrate the superiority of inclisiran compared to placebo in reducing the risk of 4P-MACE (CV death, non-fatal MI, non-fatal ischemic stroke, and urgent coronary revascularization).

### Definition of Primary Endpoint(s)

The primary endpoint of the study is the time to the first occurrence of any component of 4P-MACE, which is a composite endpoint consisting of CV death, non-fatal MI, non-fatal ischemic stroke, and urgent coronary revascularization.

The time-to-event is computed as the number of days from randomization to the onset of the primary endpoint event.

### Statistical Model, Hypothesis, and Method of Analysis

The primary statistical hypothesis to be tested is:

$${H}_{0} : {λ}_{inclisiran} / {λ}_{p l a c e b o} \geq 1 vs . {H}_{a} : {λ}_{inclisiran} / {λ}_{p l a c e b o} < 1 ,$$

where λ_{inclisiran} and λ_{placebo} are hazard rates of the first 4P-MACE in the inclisiran group and placebo group respectively. λ_{inclisiran}/λ_{placebo} is the hazard ratio (HR) of inclisiran relative to placebo.

The primary efficacy endpoint will be analyzed using a Cox proportional hazards model stratified by region and baseline status of statin treatment (yes/no), with treatment group as the only factor in the model. The estimated HR and the corresponding unadjusted two-sided 95% confidence interval from the Cox regression model will be provided separately. The primary hypothesis will be tested by a log-rank test stratified by region and baseline status of statin treatment (yes/no). The p-value for testing the above hypothesis will be provided. The superiority of inclisiran to placebo can be claimed if the p-value is less than or equal to the corresponding alpha level of the current stage.

The overall type I error rate of the study for the tests of primary and secondary endpoints will be controlled at 0.025 (one-sided). The significance level to be used for the final analysis will be adjusted to account for the alpha level spent in the interim efficacy analysis.

### Supplementary Analysis

The survival function for each treatment arm will be estimated by Kaplan-Meier method, and the Kaplan-Meier or cumulative incidence curves will be presented by treatment arm for the primary composite endpoint and its components separately.

The frequency and percentage of participants that reach the primary composite endpoint will be provided by treatment group. The components of the primary composite endpoint will also be summarized descriptively to evaluate their contributions to the overall treatment effect. The treatment comparison based on the marginal time to fatal and non-fatal MI, or fatal and non-fatal stroke, will be analyzed using the same Cox regression model as for the primary analysis.

The following supplementary analyses may also be performed for the primary endpoint:
Modification of the primary analysis using the investigator-reported primary endpoint (i.e., time to the first occurrence of investigator-reported 4P-MACE).
Modification of the primary analysis using covariate-adjusted analysis (e.g., adjusting for baseline risk prediction based on Pooled Cohort Equation).
The primary endpoint will be analyzed using a Fine-Gray sub-distribution hazard model, with non-CV death being modeled as a competing risk. The cumulative incidence functions considering non-CV death as a competing risk will also be presented.
Modification of the primary analysis where deaths due to undetermined causes are categorized as CV death.

Additional supplementary analyses may be planned and documented in the Statistical Analysis Plan (SAP) if necessary.

### Subgroup Analyses

Subgroup analyses to assess the homogeneity of the treatment effect across demographic and baseline characteristics may be performed. The subgroups under consideration include:
Age groups (<65, 65 to <75, and ≥75 years)
Gender
Race
Region
Baseline BMI (<30, 30 to <35, and ≥35 kg/m²)
Baseline eGFR (<30, 30 to <60, and ≥60 mL/min/1.73 m²)
Diabetes mellitus at baseline (Y/N)
Current smoker at baseline (Y/N)
Hypertension at baseline (Y/N)
Baseline LDL-C (<100 and ≥100 mg/dL)
Baseline Lp(a) (by median, quartile, and specific cut-offs to be determined in the SAP prior to database lock)
Baseline triglyceride (<150, 150 to <200, and ≥200 mg/dL)
Baseline hsCRP (<2 and ≥2 mg/dL)
Baseline statin intensity (high, moderate, and no statin)

The estimated HR and two-sided 95% confidence interval for each subgroup will be derived based on the Cox's proportional hazards model with treatment, subgroup, and treatment-by-subgroup as covariates. The nominal p-value for treatment by subgroup interaction will be presented as well. No adjustment for multiple comparisons will be made. Additionally, the frequency and percentage of subjects reaching the primary composite endpoint will be presented by treatment group for each subgroup.

### Secondary Endpoint(s) Estimand(s) Analysis

### Efficacy and or Pharmacodynamic Endpoint(s)

There are five secondary endpoints considered for efficacy:
Time to the first occurrence of 3P-MACE (CV death, non-fatal MI, and non-fatal ischemic stroke)
Times to the occurrences of 4P-MACE (First and recurrent CV death, non-fatal MI, non-fatal ischemic stroke, and urgent coronary revascularization)
Times to the occurrences of 3P-MACE (First and recurrent CV death, non-fatal MI, and non-fatal ischemic stroke)
Time to CV death
Time to all-cause mortality

The adjudicated endpoint of the above will be used in the analyses unless otherwise specified.

The corresponding hypotheses for the secondary endpoints are as follows:
Time to the first occurrence of 3P-MACE

$${H}_{1} : {λ}_{inclisiran} / {λ}_{p l a c e b o} \geq 1 vs . {H}_{a 1} : {λ}_{i n c l i s i r a n} / {λ}_{p l a c e b o} < 1 .$$

Times to the occurrences of 4P-MACE (first and recurrent)
   H₂: Rate ratio of inclisiran/placebo ≥1 vs. Hₐ₂: Rate ratio of inclisiran/placebo <1.
Times to the occurrences of 3P-MACE (first and recurrent)
   H₃: Rate ratio of inclisiran/placebo ≥1 vs. Hₐ₃: Rate ratio of inclisiran/placebo <1.
Time to CV death

$${H}_{4} : {λ}_{inclisiran} / {λ}_{p l a c e b o} \geq 1 vs . {H}_{a 4} : {λ}_{i n c l i s i r a n} / {λ}_{p l a c e b o} < 1 .$$

Time to all-cause mortality

$${H}_{5} : {λ}_{inclisiran} / {λ}_{p l a c e b o} \geq 1 vs . {H}_{a 5} : {λ}_{i n c l i s i r a n} / {λ}_{p l a c e b o} < 1 .$$

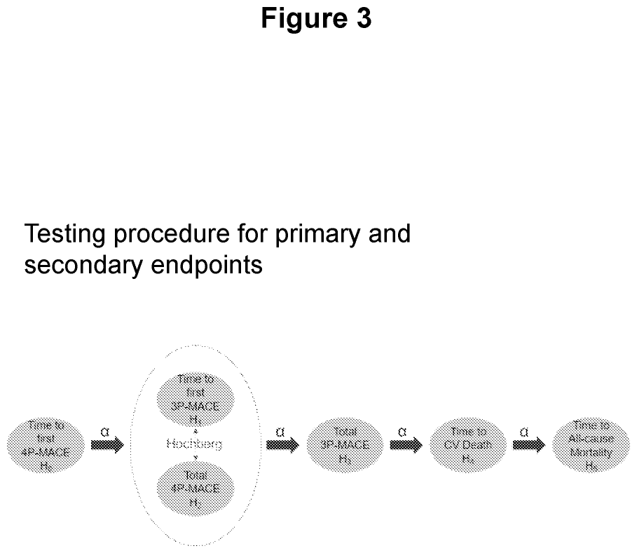

After successful rejection of the primary endpoint hypothesis (H₀), H₁ and H₂ will be tested using Benjamini-Hochberg procedure at the alpha level available at current stage. If both H₁ and H₂ are rejected, then H₃, H₄ and H₅ will be tested sequentially under hierarchical testing procedure at the alpha level available at current stage. The testing procedure to be followed is graphically presented in FIG. 3. Note: Only if both H₁ and H₂ are rejected, alpha will be passed on to test H₃.

The secondary efficacy endpoints of time to the first occurrence of 3P-MACE, time to CV death, and time to all-cause mortality will be analyzed using the same Cox regression model as for the primary efficacy endpoint as described above. The estimated HRs and the corresponding unadjusted two-sided 95% confidence intervals from the Cox regression model will be provided separately. The nominal p-values for testing H₁, H₄ and H₅ will also be provided.

The secondary efficacy endpoints of times to the occurrences of 4P-MACE and times to the occurrences of 3P-MACE will be analyzed using a semi-parametric proportional rates model (abbreviated as LWYY model) as proposed by Lin et al 2000. The estimated rate ratios and their 95% confidence intervals will be provided, as well as nominal p-values for testing the corresponding hypotheses H₂ and H₃.

### Safety Endpoints

The study will implement collection of targeted AEs, and this includes all SAEs, all AEs leading to discontinuation of study drug, and AEs of special interest (AEs of New Onset of Diabetes Mellitus in participants free from diabetes mellitus at baseline). In addition, liver function tests will be collected during follow-up until the EOS visit in participants with AST or ALT >1×ULN at screening visit (estimated to be approximately 5% of the study population) and a randomly selected subset (approximately 15% of the study population).

The on-treatment period lasts from the date of first administration of study treatment to 180 days after the date of the last actual administration of any study treatment.

### Results

This study is expected to confirm that administering inclisiran sodium 300 mg, e.g. s.c. (subcutaneous), equivalent to 284 mg inclisiran, on Day 1, Day 90, then every 6 months thereafter, is effective for reducing the risk of 4P-MACE in adult patients that have not experienced a major ASCVD event, compared to placebo.

This study is also expected to confirm that administering inclisiran sodium 300 mg, e.g. s.c. (subcutaneous), equivalent to 284 mg inclisiran on Day 1, Day 90, then every 6 months thereafter, is effective for reducing the risk of 3P-MACE (CV death, non-fatal MI, or non-fatal ischemic stroke), CV death, and all-cause mortality in adult patients that have not experienced a major ASCVD event, compared to placebo.

This study is also expected to confirm that administering inclisiran sodium 300 mg, e.g. s.c. (subcutaneous), equivalent to 284 mg inclisiran on Day 1, Day 90, then every 6 months thereafter, is effective for reducing the risk of CV death in adult patients that have not experienced a major ASCVD event, compared to placebo.

This study is also expected to confirm that administering inclisiran sodium 300 mg, e.g. s.c. (subcutaneous), equivalent to 284 mg inclisiran on Day 1, Day 90, then every 6 months thereafter, is effective for reducing the risk of all-cause mortality in adult patients that have not experienced a major ASCVD event, compared to placebo.

### REFERENCES

Achenbach S, Raggi P (2010) Imaging of coronary atherosclerosis by computed tomography; European Heart Journal, Volume 31, Issue 12, June 2010, Pages 1442-1448, https://doi.org/10.1093/eurheartj/ehq150
Benjamin E J, Muntner P, Alonso A, et al (2019) Heart Disease and Stroke Statistics—2019 Update: A report from the American Heart Association. Circulation; 139(10):e56-e528.
D'agostino RB, Vasan R S, Pencina M J, Wolf P A, Cobain M, Massaro J M, Kannel W B. General cardiovascular risk profile for use in primary care. Circulation. 2008 Feb. 12; 117:743-53. PMID:18212285.
Fitzgerald K, White S, Borodovsky A, et al (2017) A highly durable RNAi therapeutic inhibitor of PCSK9. N Engl J Med; 376(1):41-51.
Goff D C, Lloyd-Jones D M, Bennett G, et al (2014) 2013 ACC/AHA guideline on the assessment of cardiovascular risk: a report of the American College of Cardiology/American Heart Association Task Force on Practice Guidelines. Circulation; 129(25 Suppl 2):S49-73.
Grundy S M, Brewer H B, Cleeman J I, et al (2004) Definition of metabolic syndrome: Report of the National Heart, Lung, and Blood Institute/American Heart Association conference on scientific issues related to definition. Circulation; 109(3):433-8.
Grundy S M, Stone N J, Bailey A L, et al (2019) 2018 AHA/ACC/AACVPR/AAPA/ABC/ACPM/ADA/AGS/APhA/ASPC/NLA/PCNA guideline on the management of blood cholesterol: A report of the American College of Cardiology/American Heart Association Task Force on clinical practice guidelines. J Am Coll Cardiol; 73(24):3158-3209.
Leqvio SmPC (2020) Summary of Product Characteristics. Leqvio (inclisiran) 284 mg solution for injection. Novartis Europharm Limited, Dublin, Ireland. Updated 19 Aug. 2022.
Leqvio USPI (2021). Leqvio (inclisiran) 284 mg injection for subcutaneous use. US Prescribing Information. Novartis Pharmaceuticals Corporation, East Hanover, NJ. Revised December 2021.
Lin D Y, Wei L J, Yang I, et al (2000) Semiparametric regression for the mean and rate functions of recurrent events. J R Statist Soc B; 62(4):711-30.
Lloyd-Jones D M, Braun L T, Ndumele C E, Smith S C Jr, Sperling L S, Virani S S, Blumenthal R S. Use of risk assessment tools to guide decision-making in the primary prevention of atherosclerotic cardiovascular disease: a special report from the American Heart Association and American College of Cardiology; JACC November 2018, 25711; DOI:10.1016/j.jacc.2018.11.005
Mach F, Baigent C, Catapano A L, et al (2020) 2019 ESC/EAS Guidelines for the management of dyslipidaemias: lipid modification to reduce cardiovascular risk. Eur Heart J; 41(1):111-88.
Raal F J, Kallend D, Ray K K, et al (2020) Inclisiran for the treatment of heterozygous familial hypercholesterolemia. N Engl J Med; 382(16):1520-30.
Randomized Trial Assessing the Effects of Inclisiran on Clinical Outcomes Among People With Cardiovascular Disease (ORION-4). Available from: //clinicaltrials.gov/ct2/show/NCT03705234 (Accessed 29 Jun. 2022).
Ray K K, Stoekenbroek R M, Kallend D, et al (2019) Effect of 1 or 2 doses of inclisiran on low-density lipoprotein cholesterol levels: One-year follow-up of the ORION-1 randomized clinical trial. JAMA Cardiol; 4(11):1067-75.
Ray K K, Wright R S, Kallend D, et al (2020) Two phase 3 trials of inclisiran in patients with elevated LDL cholesterol. N Engl J Med; 382(16):1507-19.
Recommended INN: List 76 from the WHO Drug Information, Vol. 30, No. 3, 2016, pages 504-506, inclisiran.
Remington: The Science and Practice of Pharmacy, 23rd Edition, 2020
Study of Inclisiran to Prevent Cardiovascular (CV) Events in Participants With Established Cardiovascular Disease (VICTORION-2P). Available from: //clinicaltrials.gov/ct2/show/NCT05030428 (Accessed 29 Jun. 2022).
WHO (2019) Cardiovascular diseases (CVDs). Available from: //who.int/health-topics/cardiovascular-diseases#tab=tab_1 (Accessed 29 Jun. 2022).
Wright R S, Ray K K, Raal F J, et al (2021) Pooled patient-level analysis of inclisiran trials in patients with familial hypercholesterolemia or atherosclerosis. J Am Coll Cardiol; 77(9):1182-93.

SEQUENCES

SEQ ID NO: 1
5′-ACAAAAGCAAAACAGGUCUAGAA-3′
SEQ ID NO: 2
5′-CUAGACCUGUUUUGCUUUUGU-3′
SEQ ID NO: 3
5′-asCfsaAfAfAfgCfaAfaAfcAfgGfuCfuagsasa-3′, in which a, g, c and u are 2′-O-methyl
(2′-OMe) A, G, C, or U; Af, Gf, Cf or Uf are 2′-fluoro A, G, C or U; and s is a
phosphorothioate linkage
SEQ ID NO: 4
5′-csusagacCfuGfudTuugcuuuugu-3′, in which a, g, c and u are 2′-O-methyl (2′-OMe) A,
G, C, or U; Af, Gf, Cf or Uf are 2′-fluoro A, G, C or U; dT is 2′-deoxythymidine; and
s is a phosphorothioate linkage

1. A method of reducing the risk of a major adverse cardiovascular event (MACE) in a subject that has not experienced a major atherosclerotic cardiovascular disease (ASCVD) event,
wherein the MACE is cardiovascular death, non-fatal myocardial infarction (MI), non-fatal ischemic stroke, or urgent coronary revascularization,
wherein the method comprises administering an interfering ribonucleic acid (RNAi) agent to the subject, wherein the RNAi agent comprises:
an antisense strand comprising the nucleotide sequence 5′-asCfsaAfAfAfgCfaAfaAfcAfgGfuCfuagsasa-3′ (SEQ ID NO: 3), and
a sense strand comprising the nucleotide sequence 5′-csusagacCfuGfudTuugcuuuugu-3′ (SEQ ID NO: 4),
wherein a, g, c, and u are 2′-O-methyl (2′-OMe) modified A, G, C, and U nucleotides, respectively; Af, Gf, Cf, and Uf are 2′-fluoro A, G, C, and U modified nucleotides, respectively; dT is a deoxy-thymine nucleotide and s is a phosphorothioate linkage; and
wherein the sense strand is conjugated to at least one ligand.

2. The method of claim 1, wherein the ligand comprises the following structure:

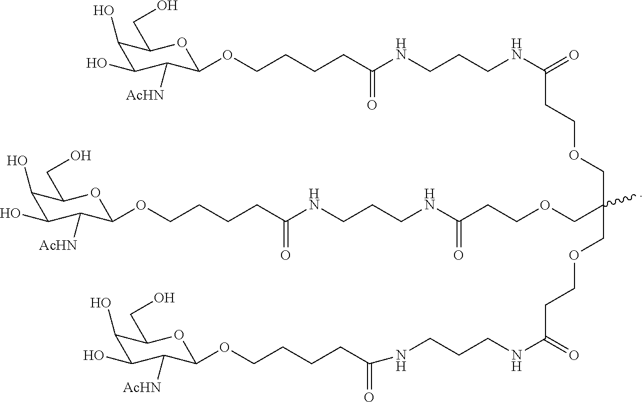 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

3. The method of claim 2, wherein the ligand is conjugated to the 3′ end of the sense strand.

4. The method of claim 3, wherein the RNAi agent is conjugated to the ligand as shown in the following schematic: 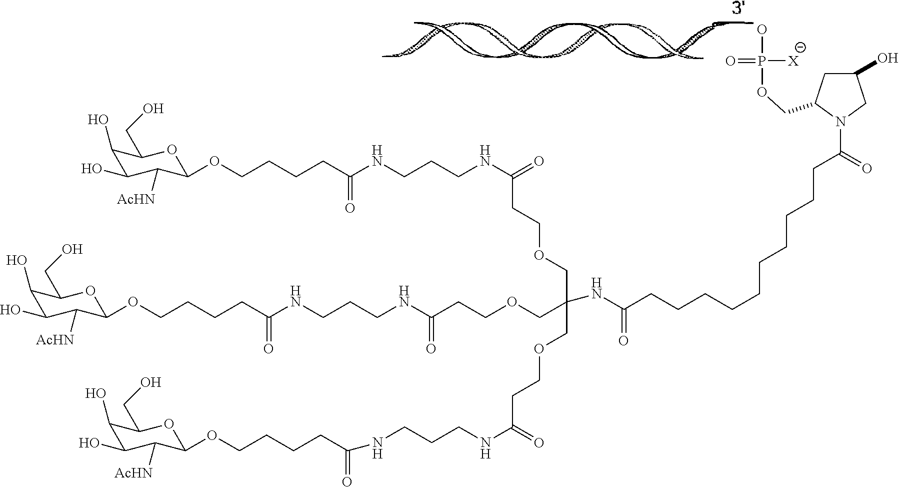
wherein X is O or S.

5. The method of claim 4, wherein X is O.

6. The method of claim 1, wherein the subject is at an increased risk for a first MACE.

7. The method of claim 6, wherein the increased risk for a first MACE is defined as any one of the following:
a) Evidence of atherosclerotic CAD on CT or invasive coronary angiogram defined as a coronary artery stenosis ≥20% but <50% in the left main coronary artery or a stenosis ≥20% but <70% in any major epicardial coronary artery;
b) Coronary artery calcium (CAC) score obtained by CT-scan ≥100 Agatston units;
c) High 10-year ASCVD risk ≥20% (assessed with the Pooled Cohort Equation);
d) Intermediate 10-year ASCVD risk 7.5%—<20% (assessed with the Pooled Cohort Equation) with at least two of the following risk-enhancing factors:
i. A first degree relative with history of premature ASCVD (males, age <55 years; females, age <65 years)
ii. History of premature menopause before age 40
iii. History of maternal pregnancy complications (defined as a history of either preeclampsia, gestational hypertension, or gestational diabetes)
iv. South Asian ancestry
v. Chronic inflammatory disorders (defined as either rheumatoid arthritis, systemic lupus erythematosus, psoriasis covering ≥10% of body surface area, Crohn's disease, ulcerative colitis, or HIV/AIDS)
vi. High-sensitivity C-reactive protein (hsCRP) ≥2 mg/L—documented in the 12 months preceding the screening visit, and, in absence of any underlying acute or inflammatory conditions (including any of the chronic inflammatory disorders listed under criteria 3 d. v.)
vii. Lipoprotein (a) ≥50 mg/dL or 125 nmol/L
viii. Estimated glomerular filtration rate (eGFR)<60 mL/min/1.73 m²
ix. Ankle branchial index (ABI)<0.9 with no symptoms of intermittent claudication; or
x. Metabolic syndrome.

8. The method of claim 7, wherein the subject has a LDL-C level ≥70 mg/dL (≥1.81 mmol/L) but <190 mg/dL (<4.91 mmol/L).

9. The method of claim 8, wherein the subject is being treated or has been treated with a background lipid-lowering therapy (LLT).

10. The method of claim 8, wherein the subject has not been or is not being treated with a background lipid-lowering therapy (LLT).

11. The method of claim 8, wherein the subject has not been treated with an additional PCSK9-targeted therapy prior to administration of the RNAi agent.

12. The method of claim 8, wherein the subject has been treated with an additional PCSK9-targeted therapy prior to administration of the RNAi agent.

13. The method of claim 1, wherein the subject has not experienced a MACE at any time prior to administration of the RNAi agent.

14. The method of claim 1, wherein the major ASCVD event is defined as any one of the following:
a. Acute coronary syndrome (ACS) at any time prior to administration of the RNAi agent, for example ACS within about 12 months prior to administration of the RNAi agent;
b. Myocardial infarction at any time prior to administration of the RNAi agent;
c. Ischemic stroke at any time prior to administration of the RNAi agent; or
d. Symptomatic peripheral artery disease (PAD), for example as evidenced by either intermittent claudication, previous revascularization, or amputation due to atherosclerotic disease, at any time prior to administration of the RNAi agent.

15. The method of claim 14, wherein the subject
(i) does not have a history of ischemia-driven revascularization in a coronary or extracoronary arterial bed prior to administration of the RNAi agent;
(ii) has an absence of coronary atherosclerosis on a CT angiogram or an invasive coronary angiogram in the 2 years prior to administration of the RNAi agent;
(iii) has not had a coronary artery calcium (CAC) score of 0 obtained in the 2 years prior to administration of the RNAi agent; and/or
(iv) does not have active liver disease or hepatic dysfunction, for example identified as having aspartate aminotransferase (AST) or alanine aminotransferase (ALT) greater than or equal to 3 times the upper limit of normal (ULN).

16-18. (canceled)

19. The method of claim 18, wherein the subject is on a diet designed to improve lipid levels, a maximally tolerated statin therapy, and/or a low-density lipoprotein lowering therapy.

20. The method of claim 19, wherein the administration of the RNAi agent reduces the low-density lipoprotein cholesterol (LDL-C) level in the subject.

21-29. (canceled)

30. The method of claim 1, wherein the RNAi agent is administered initially, again at 3 months, and then every 6 months.

31. (canceled)

32. The method of claim 1, wherein the method comprises administering the RNAi agent subcutaneously (for example, into the abdomen, upper arm, or thigh).

33-36. (canceled)

37. The method of claim 1, wherein the RNAi agent is administered in a sterile aqueous solution, for example in a volume of about 1.5 mL.

38. The method of claim 37, wherein the sterile aqueous solution contains sodium hydroxide and/or phosphoric acid.

39. The method of claim 38, wherein the sterile aqueous solution has a pH of about 7.0.

40. The method of claim 37, wherein the sterile aqueous solution is contained in a prefilled syringe for subcutaneous administration.

41. The method of claim 1, wherein the RNAi agent is inclisiran.

42. (canceled)

43. The method of claim 41, wherein each dose comprises about 284 mg of inclisiran (equivalent to about 300 mg of inclisiran sodium).

44. The method of claim 43, wherein a dose of 284 mg of inclisiran (equivalent to about 300 mg of inclisiran sodium) is administered as a single subcutaneous injection initially, again at 3 months, and then every 6 months.

45-46. (canceled)


SEQUENCE LISTING (4 sequences)

SEQ ID NO: 1 - RNA, 23 nt, synthetic construct
  source 1..23: mol_type = other RNA
acaaaagcaaaacaggtctagaa

SEQ ID NO: 2 - RNA, 21 nt, synthetic construct
  source 1..21: mol_type = other RNA
ctagacctgttttgcttttgt

SEQ ID NO: 3 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-fluorocytidine-3'-phosphorothioate
  modified_base 3: mod_base = OTHER; note = 2'-O-methyladenosine-3'-phosphate
  modified_base 4: mod_base = OTHER; note = 2'-fluoroadenosine-3'-phosphate
  modified_base 5: mod_base = OTHER; note = 2'-fluoroadenosine-3'-phosphate
  modified_base 6: mod_base = OTHER; note = 2'-fluoroadenosine-3'-phosphate
  modified_base 7: mod_base = gm
  modified_base 8: mod_base = OTHER; note = 2'-fluorocytidine-3'-phosphate
  modified_base 9: mod_base = OTHER; note = 2'-O-methyladenosine-3'-phosphate
  modified_base 10: mod_base = OTHER; note = 2'-fluoroadenosine-3'-phosphate
  modified_base 11: mod_base = OTHER; note = 2'-O-methyladenosine-3'-phosphate
  modified_base 12: mod_base = OTHER; note = 2'-fluoroadenosine-3'-phosphate
  modified_base 13: mod_base = cm
  modified_base 14: mod_base = OTHER; note = 2'-fluoroadenosine-3'-phosphate
  modified_base 15: mod_base = gm
  modified_base 16: mod_base = OTHER; note = 2'-fluoroguanosine-3'-phosphate
  modified_base 17: mod_base = um
  modified_base 18: mod_base = OTHER; note = 2'-fluorocytidine-3'-phosphate
  modified_base 19: mod_base = um
  modified_base 20: mod_base = OTHER; note = 2'-O-methyladenosine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methyladenosine
acaaaagcaaaacaggtctagaa

SEQ ID NO: 4 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyluridine-3'-phosphorothioate
  modified_base 3: mod_base = OTHER; note = 2'-O-methyladenosine-3'-phosphate
  modified_base 4: mod_base = gm
  modified_base 5: mod_base = OTHER; note = 2'-O-methyladenosine-3'-phosphate
  modified_base 6: mod_base = cm
  modified_base 7: mod_base = OTHER; note = 2'-fluorocytidine-3'-phosphate
  modified_base 8: mod_base = um
  modified_base 9: mod_base = OTHER; note = 2'-fluoroguanosine-3'-phosphate
  modified_base 10: mod_base = um
  modified_base 11: mod_base = OTHER; note = 2'-deoxythymidine-3'-phosphate
  modified_base 12: mod_base = um
  modified_base 13: mod_base = um
  modified_base 14: mod_base = gm
  modified_base 15: mod_base = cm
  modified_base 16: mod_base = um
  modified_base 17: mod_base = um
  modified_base 18: mod_base = um
  modified_base 19: mod_base = um
  modified_base 20: mod_base = gm
  modified_base 21: mod_base = um
ctagacctgttttgcttttgt